(19) United States
(12) Patent Application Publication
(10) Pub. No.: US 2003/0144204 A1
(43) Pub. Date: Jul. 31, 2003

(54) Akt-based inducible survival switch

(75) Inventor: David Spencer, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine

(21) Appl. No.: 10/324,985

(22) Filed: Dec. 19, 2002

Related U.S. Application Data
(60) Provisional application No. 60/342,155, filed on Dec. 19, 2001.

(51) Int. Cl. A61K 48/00; C07K 14/705; A61K 38/17; C07H 21/04

(52) U.S. Cl. 514/12; 514/44; 530/350; 536/23.5; 435/320.1; 435/325

(57) ABSTRACT

The present invention relates to the field of apoptosis and programmed cell-death. More particularly, it relates to expression vectors, pharmaceutical compositions and methods for inhibiting cell-death using the expression vectors and/or pharmaceutical compositions. Yet further, the present invention also relates to methods of using the expression vector to screen for additional regulators of an anti-apoptotic gene.

This application claims priority to U.S. Provisional Application serial No. 60/342,155 filed on Dec. 19, 2001.

This invention was made with government support under NIH Grant Nos. R01-CA87569 and U01-CA84296 awarded by the National Institutes of Health. The United States Government may have certain rights in the invention.

### BACKGROUND

I. Field of Invention

The present invention relates to the field of apoptosis and programmed cell-death. More particularly, it relates to pharmaceutical compositions and methods for inhibiting cell-death.

II. Related Art

Programmed cell-death (also known as apoptosis) is a form of cell-death defined by morphological and biochemical characteristics. Apoptosis is a characteristic of the normal developmental process as well as a response of cells to stress or other environmental insults. Apoptosis is characterized by membrane blebbing and retention of its integrity, cellular and cytoplasmic shrinkage, chromosome fragmentation and condensation, and endonuclease activation resulting in the characteristic 180 bp DNA ladder. During this process, the nuclear lamins are cleaved inducing their disassembly. Apoptosis does not induce an inflammatory response because cells form apoptotic bodies, which are phagocytosed by neighboring cells. Uptake of apoptotic cells may also convert dendritic cells to an anti-inflammatory state (Fadok VA, 2000). A number of stresses can induce apoptosis in vitro and in vivo. The administration of gluccocorticoids, reduction of hormone and/or growth factor levels, chemotherapy (toxic agents), mechanical injury and DNA damage can all result in apoptosis. Apoptosis is also induced by aberrant cell cycle activity, and it can be triggered in cells that express the Fas receptor with cross-linking antibodies or the natural Fas ligand. High frequencies of apoptotic cell-death are associated in a diverse array of pathological disorders.

When growth factors are limiting in the extracellular milieu, most cell types die by apoptosis due to finely tuned homeostatic mechanisms. One common pathway by which ligand-bound growth factor receptors prevent apoptosis is through the phosphorylation-dependent membrane recruitment and activation of phosphatidylinositol 3-kinases (PI3K) PI3Ks generate phosphatidylinositol 3,4-diphosphate (PtdIns(3,4)P₂) and PtdIns(3,4,5)P₃ by phosphorylating the D-3 position of the inositol ring of phosphoinositides. In turn, these 3-phosphorylated lipids can lead to the plasma membrane recruitment and activation of a number of cytosolic signaling molecules by binding to their pleckstrin homology (PH) domains. The importance of the PH is underlined by its recent discovery in over 250 genes, thus the PH domain is the 11th most common InterPro family found in the human proteome (Lander, 2001). Although the cellular responses regulated by PI3Ks are diverse, including growth, survival, transformation, vesicle trafficking, and others (Wymann et al., 1998), activation of the serine/threonine kinase Akt/CAKT, (the cellular homologue of the viral oncogene, v-Akt), appears to be central to the PI3K-mediated delay of apoptosis and increase of cell survival (Chan, 1999).

Although c-Akt was cloned a decade ago (Bellacosa et al., 1991), the mechanism by which Akt propagates survival signals in eukaryotic cells has only been elucidated more recently (Datta et al., 1999). All three mammalian isoforms of Akt (Akt1/PKBα/RAC-PKα, Akt2/PKBβ/RAC-PKβ, and Akt3/RAC-PKγ) have an amino-terminal PH domain, a serine-threonine (S/T) kinase domain related to protein kinase A and C (PKA and PKC) family members, and a carboxy-terminal regulatory domain. Akt is activated in response to various survival stimuli, such as growth factors, cytokines and hormones, in a PI3K-dependent manner (Frank et al., 1995). In addition, PI3K independent activation of Akt has also been shown after treatment with heat shock (Shaw et al., 1998), α-adrenergic receptor activation (Zhu et al., 2001), PKC activation (Kroner et al., 2000) and c-AMP upregulation (Filippa et al., 1999). It is believed that Akt activation involves three steps, in which the first step is the interaction of the inhibitory PH domain with PtdIns(3,4)P₂ and PtdIns(3,4,5)P₃ leading to membrane recruitment and a conformational change in the kinase. Together these two events expose T308 (based on Akt1) in the activation loop of the catalytic domain to the constitutively active, PtdIns(3,4,5)P₃-dependentkinase-1 (PDK1). Finally, T308 phosphorylation leads to phosphorylation in the regulatory domain at S473 (Akt1) by PDK2. Although PDK2 is still poorly defined, PKC members, integrin-linked kinase (ILK), PDK1 (bound to PRK2 (Balendran et al., 1999), and Akt autophosphorylation (Toker et al., 2000) have all been reported to be the effectors of this event (Chan et al., 1999; Bellacosa, et al., 1991).

Further underlying the nodal position of Akt in survival signaling are the observations that pT308 and pS473 have a relatively short half-life in vivo, and phosphatase inhibitors, such as calyculin A and okadaic acid, a relatively specific inhibitor of PP2A, are able to prevent Akt dephosphorylation and inactivation (Meier et al., 1998). Moreover, Akt family members are upregulated in several cancers and inactivation of the PtdIns phosphatase, PTEN, is also associated with cancer and Akt activation (Nakatani et al., 1999; Yuan et al., 2000; Liu et al., 1998; Stambolic et al., 1998; Li et al., 1998; Cantley et al., 1999).

To date, Akt has been implicated in various physiological processes including cell cycle regulation, cellular metabolism and cell survival. The first identified downstream target of Akt was glycogen synthase kinase-3 (GSK-3), which is phosphorylated at serine 21 in GSK3-α and serine 9 in GSK3-β, leading to inactivation and the upregulation of a number of substrates involved in cellular metabolism, including glycogen synthesis (Cross et al., 1995). Recently, several targets of the PI3K/Akt signaling pathway have been identified that may explain the ability of this regulatory cascade to promote survival (Datta et al., 1999). These targets include two components of the intrinsic cell death machinery, Bad and caspase 9, transcription factors of the forkhead family (i.e., AFX) that can upregulate FasL, and the kinase, IKKα, that regulates the anti-apoptotic transcription factor, NF-κB. Additional substrates for Akt include eNOS, phosphofructokinase-2, phosphodiesterase 3β, and the reverse transcriptase subunit of telomerase. These and other as-yet-unidentified Akt substrates might mediate the effects of Akt on cellular survival.

In order to elucidate the function of many signaling molecules, constitutively active or “dominant negative” mutant proteins are often overexpressed in target cells. When Akt or many other “upstream” signaling molecules are modified to contain a membrane-targeting sequence, the increased proximity to activating kinases, such as PDK1, or to membrane-localized substrates typically leads to the constitutive phenotype. For example, most functional Akt studies have utilized either Src family myristoylation-targeting peptides or the myristoylated gag sequence within v-Akt. Under these conditions, however, the kinase is activated as soon as it is expressed in cells, but the effects of activation may not be monitored until much later, when the direct effects of Akt are typically obscured. For controlled gene expression or kinase activation, several approaches are available such as tetracycline-regulatable transcription systems (Gossen et al., 1995), chimeras of hormone binding domains (HBD) with target proteins (Jackson et al., 1993; Picard, 1994; Samuels et al., 1993) and chemically induced dimerization (CID) (Spencer et al., 1993; Spencer, 1996).

The present invention provides the first CID regulatable anti-apoptotic gene, Akt. Thus, as gene therapy comes of age, the ability to conditionally regulate viability with an anti-apoptotic “survival switch” like inducible Akt is likely to be as welcome as the more well established pro-apoptotic suicide genes.

### BRIEF SUMMARY OF THE INVENTION

The present invention has developed an Akt molecule, inducible Akt (iAkt), whose range of activation extends from undetectable to comparable to that of constitutively active Myr-Akt. Activation of iAkt is based on ligand-dependent recruitment of chimeric Akt to a membrane-bound myristoylated “docking protein”. It is envisioned that the Akt molecules of the present invention can be used to inhibit apoptotic cell-death and to treat conditions, such as myocardial infarction and hyperproliferative diseases, which result in increased apoptotic cell-death. Moreover, it is envisioned that the Akt molecules of the present invention can be used to decrease cell-death during tissue and/or organ transplantation.

A specific embodiment of the present invention is an expression vector encoding an inducible chimeric protein comprising a mutant Akt polypeptide fused to a ligand-binding domain, for example a derivative of FKBP. The mutant Akt can lack a pleckstrin homology domain. Yet further, the expression vector can comprise more than one ligand-binding domain. The expression vector can be admixed with a pharmaceutically acceptable carrier resulting in a pharmaceutical composition. In further embodiments, the expression vector is used to transform host cells.

Another embodiment is a fusion protein comprising a mutant Akt sequence and at least one ligand-binding domain, for example a derivative of FKBP. The mutant Akt can lack a pleckstrin homology domain. The fusion protein can be admixed with a pharmaceutically acceptable carrier resulting in a pharmaceutical composition.

Yet further, another embodiment of the present invention is a method of modulating apoptosis comprising the steps of: administering to a cell susceptible to apoptosis an expression vector encoding an inducible chimeric protein comprising a mutant Akt polypeptide fused to a ligand-binding domain; administering to the cell a second expression vector encoding a second ligand-binding domain fused to a membrane-targeting region; and modulating apoptosis by administering to the cell a chemical ligand, wherein the ligand results in activation of the mutant Akt. In specific embodiments, the first ligand-binding domain is a derivative of FKBP and the second ligand-binding domain is a rapamycin binding domain from mTOR/FRAP/RAFT. The chemical ligand is a rapamycin analog and the membrane-targeting region can be a myristoylated target sequence. Yet further, an anti-apoptotic agent or a suicide gene can be administered to the genetically engineered cell.

Another embodiment is a method of modulating apoptosis comprising the steps of: administering to a cell susceptible to apoptosis an expression vector encoding an inducible chimeric protein comprising a mutant Akt polypeptide fused to a ligand-binding domain and a second chimeric protein comprising a ligand-binding domain fused to a membrane-targeting region; and modulating apoptosis by administering to the cell a chemical ligand, wherein the ligand results in activation of the mutant Akt. The inducible chimeric protein and the second chimeric protein can be separated by an internal ribosome entry sequence or can be under transcriptional control of two promoters.

A specific embodiment is a method of modulating apoptosis in a cell susceptible to apoptosis comprising the steps of administering a fusion protein comprising a mutant Akt sequence and at least one ligand-binding domain, for example a derivative of FKBP, administering a second fusion protein, wherein the second fusion protein comprises a second ligand-binding domain fused to a membrane-targeting region; and modulating apoptosis by administering to the cell a chemical ligand, wherein the chemical ligand results in activation of the mutant Akt.

A further embodiment is a method of modulating hypoxia-induced apoptosis comprising the steps of: administering to a cell suspected of hypoxia-induced apoptosis an expression vector encoding an inducible chimeric protein comprising a mutant Akt polypeptide fused to a ligand-binding domain; administering to the cell a second expression vector encoding a second ligand-binding domain fused to a membrane-targeting region; and modulating hypoxia-induced apoptosis by administering to the cell a chemical ligand, wherein the chemical ligand results in activation of the mutant Akt. Hypoxia-induced apoptosis is induced via ischemia.

Still yet, another embodiment is a method of modulating a cell suspected of hypoxia-induced apoptosis comprising the steps of administering a fusion protein comprising a mutant Akt sequence and at least one ligand-binding domain, for example a derivative of FKBP, administering a second fusion protein, wherein the second fusion protein comprises a second ligand-binding domain fused to a membrane-targeting region; and modulating hypoxia-induced apoptosis by administering to the cell a chemical ligand, wherein the chemical ligand results in activation of the mutant Akt.

Another embodiment is a method of modulating tissue damage following ischemia-reperfusion comprising the steps of: administering to a tissue suspected of tissue damage an expression vector encoding an inducible chimeric protein comprising a mutant Akt polypeptide fused to a ligand-binding domain; administering to the tissue a second expression vector encoding a second ligand-binding domain fused to a membrane-targeting region; and modulating tissue damage by administering to the tissue a chemical ligand, wherein the ligand results in activation of the mutant Akt. More specifically, the tissue is cardiac.

A further embodiment is a method of modulating tissue damage following ischemia-reperfusion comprising the steps of administering to a tissue suspected of tissue damage a fusion protein comprising a mutant Akt sequence and at least one ligand-binding domain, for example a derivative of FKBP, administering to the tissue a second fusion protein, wherein the second fusion protein comprises a second ligand-binding domain fused to a membrane-targeting region; and modulating tissue damage by administering to the cell a chemical ligand, wherein the chemical ligand results in activation of the mutant Akt.

Another embodiment is a method of treating myocardial infarction comprising the step of: administering to a subject in need of such treatment an inducible Akt molecule in an amount effective to reduce cardiac tissue necrosis in the subject.

Yet further, another embodiment is a method of modulating tissue damage during transplantation comprising the steps of: administering to a tissue suspected of tissue damage an expression vector encoding an inducible chimeric protein comprising a mutant Akt polypeptide fused to a ligand-binding domain; administering to the tissue a second expression vector encoding a second ligand-binding domain fused to a membrane-targeting region; and modulating tissue damage by administering to the tissue a chemical ligand, wherein the chemical ligand results in activation of the mutant Akt.

A specific embodiment of the present invention is a method of modulating tissue damage following ischemia-reperfusion comprising the steps of administering to a tissue suspected of tissue damage a fusion protein comprising a mutant Akt sequence and at least one ligand-binding domain, for example a derivative of FKBP, administering to the tissue a second fusion protein, wherein the second fusion protein comprises a second ligand-binding domain fused to a membrane-targeting region; and modulating tissue damage by administering to the cell a chemical ligand, wherein the chemical ligand results in activation of the mutant Akt.

Another embodiment is a method of screening compounds to identify a modulator of Akt comprising the steps of: providing a cell expressing iAkt; contacting the cell with a candidate compound; admixing rapamycin analogs to induce activation of Akt; measuring the level of activation of Akt; and comparing the Akt activation in the presence of the candidate compound with the activation of Akt in the absence of the candidate compound; wherein a difference in the activation of Akt in the presence of the candidate compound, as compared with the activation of Akt in the absence of the candidate compound, identifies the candidate compound as a modulator of Akt activation. Yet further, a specific embodiment can include screening compounds to identify a candidate compound that can destablize the endogenous Akt expression.

Still further, another embodiment is a method of screening compounds to identify a modulator of Akt comprising the steps of: providing a cell expressing iAkt; contacting the cell with a candidate compound; admixing rapamycin analogs to induce activation of Akt; measuring the level of phosphorylation of Akt; and comparing the Akt phosphorylation in the presence of the candidate compound with the Akt phosphorylation in the absence of the candidate compound; wherein a difference in the phosphorylation of Akt in the presence of the candidate compound, as compared with the phosphorylation of Akt in the absence of the candidate compound, identifies the candidate compound as a modulator of Akt phosphorylation.

Another embodiment of the present invention is a method of screening compounds to identify a modulator of Akt comprising the steps of: providing a cell expressing iAkt; contacting the cell with a candidate compound; admixing rapamycin analogs to induce activation of Akt; measuring Akt activity; and comparing the Akt activity in the presence of the candidate compound with the Akt activity in the absence of the candidate compound; wherein a difference in the activity of Akt in the presence of the candidate compound, as compared with the activity of Akt in the absence of the candidate compound, identifies the candidate compound as a modulator of Akt activity. It is contemplated that screening methods can also be used to identify tissue-specific Akt substrates that may provide more specific drug targets for cancer and other hyperproliferative diseases.

Yet further, another embodiment of the present invention is a method of treating a disease by screening compounds to identify a modulator of Akt comprising the steps of: providing a cell expressing iAkt; contacting the cell with a candidate compound; admixing rapamycin analogs to induce activation of Akt; measuring Akt activity; comparing the Akt activity in the presence of the candidate compound with the Akt activity in the absence of the candidate compound; wherein a difference in the activity of Akt in the presence of the candidate compound, as compared with the activity of Akt in the absence of the candidate compound, identifies the candidate compound as a modulator of Akt activity; and administering to a subject suffering from the disease the modulator of Akt activity.

Specifically, the disease is hyperproliferative disease. Exemplary hyperproliferative diseases are selected from the group consisting of rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (e.g., adenomatous hyperplasia and prostatic intraepithelial neoplasia), carcinoma in situ, oral hairy leukoplakia, and psoriasis.

Yet further, the hyperproliferative disease is further defined as cancer. Exemplary cancers are selected from the group consisting of melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, and colon cancer.

The foregoing has outlined rather broadly the features and technical advantages of the present invention in order that the detailed description of the invention that follows may be better understood. Additional features and advantages of the invention will be described herein after which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1A, FIG. 1B and FIG. 1C show a schematic representation of constructs used in this study. FIG. 1A shows the heterodimeric (HED)CIDs/rapalogs and CID_{HED}-binding domains. FIG. 1B shows that the CID-binding domains were subcloned as monomers (FRB1), dimers (FRB1 2), or tandem trimers (F3) into expression vectors to generate chimeric proteins. The c-Src myristoylation (M) signal sequence (horizontal bars) was fused to the N-terminus of FRB₁ or Akt kinase alleles (vertical dashes). Wild type or PH domain (striped) deletion mutants of Akt were fused to F3 at their N- or C-terminal ends. FIG. 1C shows the model of CID_{HED}-mediated membrane-targeting an activation of inducible Akt (iAkt) kinase.

FIG. 2 shows M-Akt enhances NF-κB induction induced by PMA. Jurkat-TAg cells were cotransfected with NF-κB/SEAP reporter plasmid along with control vector or M-Akt expression vector by electroporation.

FIG. 3A and FIG. 3B show that the CID-mediated membrane-targeting of PH.Akt, but not wild-type Akt, induces titratable NF-κB transactivation. FIGS. 3A and 3B show Jurkat-TAg cells that were transiently cotransfected with reporter plasmid NF-κB/SEAP along with (FIG. 3A) M-FRB₁2 (open circle), F3-Akt (open square), F3-Akt.KM (triangle), M-FRB₁2+F3-Akt (closed circle), M-FRB₁ 2+F3-Akt.KM (closed square), or (FIG. 3B) M-FRB1 2 (open circle), F3-APH.Akt (open square), M-FRB₁2+F3-APH.Akt (closed circle), M-Akt (closed square), or M-ΔPH.Akt (triangle).

FIG. 4A, FIG. 4B and FIG. 4C show optimization of iAkt based on CID-mediated NF-κB induction. FIGS. 4A-4C show Jurkat-TAg cells that were transiently cotransfected with NF-κB/SEAP along with (FIG. 4A) M-FRB₁ 2 (open circle), ΔPH.Akt-F3 (open square), F3-ΔPH.Akt (open triangle), M-FRB₁ 2+ΔPH.Akt-F3 (closed square), M-FRB₁ 2+F3-ΔPH.Akt (closed triangle), M-ΔPH.Akt (closed circle), or (FIG. 4B) F3-ΔPH.Akt (closed circle), M-FRB₁2 (open circle), M-FRB₁ (square), M-FRB₁ 2+F3-ΔPH.Akt(closed triangle), M-FRB₁+F3-ΔPH.Akt (open triangle), or (FIG. 4C) M-FRB₁2 (triangle), F3-ΔPH.Akt (open square), iAktₐ (open circle), iAkt_{b} (closed circle), or M-ΔPH.Akt (closed square).

FIG. 5A and FIG. 5B show phosphorylation and activation of iAkt following CID-mediated membrane-targeting. FIG. 5A shows that 293T cells were cotransfected with M-FRB₁ 2 plus F3-ΔPH.Akt followed by serum starvation. Thereafter, cells were treated and Western blot analysis was performed. FIG. 5B shows Jurkat.iAkt cells that were serum-starved for followed by treatment.

FIG. 6A, FIG. 6B and FIG. 6C show NF-κB transactivation induced by CID-mediated iAkt activation is PI3K independent. FIGS. 6A-6B show Jurkat-TAg cells that were transfected with bicistronic construct iAkt_{b} and the NF-κB/SEAP reporter. Cells transfected with M-FRB₁ 2 served as a negative control. Cells were treated with AP22783 in PMA containing media plus or minus PI3K inhibitors, wortmannin (FIG. 6A) or LY294002 (FIG. 6B). FIG. 6C shows that PI3K inhibitors prevent activation of c-Akt, but not iAkt.

FIG. 7A and FIG. 7B show that CID-mediated activation of Akt kinase blocks staurosporine (STS)-induced caspase-3activation, PARP cleavage, and apoptosis. FIG. 7A shows Jurkat.iAkt cells that were treated with STS with or without (control) AP22783 in serum-free conditions. Hypodiploid/apoptotic cells were determined by flowcytometry after PI staining of permeabilized cells. FIG. 7B shows Jurkat.iAkt cells that were treated with different doses of STS with or without AP22783. Caspase-3 activation and PARP were determined by Western blotting.

FIG. 8A, FIG. 8B, show that CID-mediated activation of Akt kinase blocks apoptosis triggered by multiple stimuli. Jurkat.iAkt cells were treated with (FIG. 8A) wortmannin, (FIG. 8B) LY294002, (FIG. 8C) anti-Fas antibody, or (FIG. 8D) etoposide with or without AP22783. Apoptotic cells were measured by flow cytometry for subdiploid populations after PI staining.

### DETAILED DESCRIPTION OF THE INVENTION

It is readily apparent to one skilled in the art that various embodiments and modifications can be made to the invention disclosed in this Application without departing from the scope and spirit of the invention.

As used herein, the use of the word “a” or “an” when used in conjunction with the term “comprising” in the sentences and/or the specification may mean “one,” but it is also consistent with the meaning of “one or more,” “at least one,” and “one or more than one.”

As used herein, the term “Akt molecule”, embraces “Akt nucleic acids”, “Akt polypeptides” and/or Akt expression vectors. Yet further, it is understood the activity of Akt as used herein is driven by CID. Thus, activation of the Akt of the present invention is based on ligand-dependent recruitment of chimeric Akt to a membrane-bound (e.g., myristoylated) “docking protein”.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is preferred, such as where the non-coding regions are required for optimal expression or where non-coding regions such as introns are to be targeted in an antisense strategy.

As used herein, the term “cell” is intended to refer to a single cell or more than one cell. The cell may be in an in vivo or in vitro environment. For example, the cell can be contained within the animal or may be isolated from the animal.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat hypoxia-induced apoptosis, tissue damage from such conditions including, but not limiting to ischemia/reperfusion, myocardial infarction, organ transplantation hyperproliferative diseases, thus the expression construct or transgene is a therapeutic construct or a prophylactic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra.

As used herein, the term “functionally equivalent”, refers to Akt nucleic acid fragment, variant, mutant or analog, refers to a nucleic acid that codes for an Akt polypeptide that inhibits apoptotic cell-death of cells. Preferably the Akt polypeptide maintains a serine-threonine kinase activity. More specifically, “functionally equivalent” refers to an Akt polypeptide that has a serine-threonine kinase activity and is capable of enhancing survival of a cell that may undergo apoptotic cell-death. Thus, one of skill in the art understands that a mutant Akt molecule in the present invention is a functional equivalent of Akt.

As used herein, the term “gene” is defined as a functional protein, polypeptide, or peptide-encoding unit. As will be understood by those in the art, this functional term includes genomic sequences, cDNA sequences, and smaller engineered gene segments that express, or is adapted to express, proteins, polypeptides, domains, peptides, fusion proteins, and mutants.

As used herein, the term, “mutant Akt”, refers to an Akt molecule that has been altered, for example it lacks a functional pleckstrin homology domain. The mutant or altered Akt molecule comprises a nucleic acid molecule which hybridizes under stringent conditions to a nucleic acid having the sequence of a known Akt gene and codes for an Akt polypeptide. Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture. Yet further, “iAkt” refers to an inducible Akt molecule.

As used herein, the term “pharmaceutically or pharmacologically acceptable” refer to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “stem cells” refers to “undifferentiated” cells capable of proliferation, self-maintenance, production of differentiated cells or regeneration of stem cells.

As used herein, the term “under transcriptional control” or “operatively linked” is defined as the promoter is in the correct location and orientation in relation to the nucleic acid to control RNA polymerase initiation and expression of the gene.

The present invention is an Akt molecule, inducible Akt (iAkt), whose range of activation extends from undetectable to comparable to that of constitutively active Myr-Akt. Activation of iAkt is based on ligand-dependent recruitment of chimeric Akt to a membrane-bound myristoylated “docking protein”. Thus a conditional activation of iAkt leads to reversible protection from a number of apoptotic stimuli, including, but not limiting to the PI3K inhibitors, protein kinase inhibitors, topoisomerase inhibitors, and Fas crosslinking.

I. Engineering Expression Constructs

The present invention involves an expression vector encoding a chimeric protein a mutant Akt polypeptide and a ligand-binding domain, all operatively linked. In specific embodiments, the ligand-binding domain is a derivative of FKBP, e.g., FK506-Binding Protein.

In the present invention, Akt molecules are capable of inhibiting apoptotic cell-death both in vivo and in vitro. The Akt molecule comprises a nucleic acid molecule which: (1) hybridizes under stringent conditions to a nucleic acid having the sequence of a known Akt gene and (2) codes for an Akt polypeptide. Preferably the Akt polypeptide maintains a serine-threonine kinase activity.

Yet further, it is contemplated that other normal or mutant or altered variants of Akt can be used in the present invention. Exemplary polynucleotide sequences that encode Akt polypeptides include, but are not limited to SEQ.ID.NO:1 (GenBank accession # X65687: mouse Akt1), SEQ.ID.NO:2 (GenBank accession # U22445: mouse Akt2), SEQ.ID.NO:3 (GenBank accession # M95936: human Akt2), SEQ.ID.NO:4 GenBank accession # AF135794: human Akt3), and Akt isoforms from other species, including oncogenic viral sequences.

In certain embodiments, the present invention involves the manipulation of genetic material to produce expression constructs that encode mutants of Akt. Such methods involve the generation of expression constructs containing, for example, a heterologous nucleic acid sequence encoding mutant Akt of interest and a means for its expression, replicating the vector in an appropriate helper cell, obtaining viral particles produced therefrom, and infecting cells with the recombinant virus particles.

Thus, the preferable Akt molecule of the present invention comprises a mutated pleckstrin homology domain (PH). In specific embodiments, the PH domain is truncated or removed. It is also contemplated that the PH domain can be mutated using standard mutagenesis, insertions, deletions, or substitutions to produce an Akt molecule that does not have a functional PH domain. The preferred Akt nucleic acid has the nucleic acid sequence of SEQ.ID.NO. 5. The Akt nucleic acids of the invention also include homologs and alleles of a nucleic acid having the sequence of SEQ.ID.NO. 5, as well as functionally equivalent fragments, variants, and analogs of the foregoing nucleic acids.

In the context of gene therapy, the gene will be a heterologous polynucleotide sequence derived from a source other than the viral genome, which provides the backbone of the vector. The gene is derived from a prokaryotic or eukaryotic source such as a bacterium, a virus, yeast, a parasite, a plant, or even an animal. The heterologous DNA also is derived from more than one source, i.e., a multigene construct or a fusion protein. The heterologous DNA also may include a regulatory sequence, which is derived from one source and the gene from a different source.

In further embodiments, expression constructs are produced that contain a second chimeric protein that is essential for activation of the Akt construct. The second chimeric protein includes, but is not limited to a ligand-binding domain and a membrane-targeting region. In specific embodiments, the ligand-binding domain of the second chimeric protein is heterologous to the ligand-binding domain in the Akt construct. In a further specific embodiment, the ligand-binding domain is a rapamycin-binding domain, FRB, from FRAP/mTOR. The second chimeric protein contains a membrane-targeting domain, exemplary membrane-targeting domains include, but are not limited to a myristoylated targeting sequence, CAAX Box (prenylation targeting sequence), transmembrane anchor sequence, or other membrane-targeting regions that are well known and used in the art.

Yet further, one skilled in the art is aware that the polynucleotide sequences for the second chimeric protein can be included in the Akt expression vector in tandem under control of a separate promoter or separated by an internal ribosome entry sequence, which results in a bicistronic construct. A. Chemically Induced Dimerization

In certain embodiments, the present invention utilizes the technique of chemically induced dimerization (CID) to produce a conditionally controlled protein or polypeptide. In addition to this technique being inducible, it also is reversible, due to the degradation of the labile dimerizing agent or administration of a monomeric competitive inhibitor.

CID system uses synthetic bivalent ligands to rapidly crosslink signaling molecules that are fused to ligand-binding domains CID. This system has been used to trigger the oligomerization and activation of cell surface (Spencer et al., 1993; Spencer et al., 1996; Blau et al., 1997), or cytosolic proteins (Luo et al., 1996; MacCorkle et al., 1998), the recruitment of transcription factors to DNA elements to modulate transcription (Ho et al., 1996; Rivera et al., 1996) or the recruitment of signaling molecules to the plasma membrane to simulate signaling (Spencer et al., 1995; Holsinger et al., 1995).

The CID system is based upon the notion that surface receptor aggregation effectively activates downstream signaling cascades. In the simplest embodiment, the CID system uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs and a myristoylation sequence to the cytoplasmic signaling domain of a target receptor, one can stimulate signaling in a dimerizer drug-dependent, but ligand and ectodomain-independent manner. This provides the system with temporal control, reversibility using monomeric drug analogs, and enhanced specificity. The high affinity of third-generation AP20187/AP1903 CIDs for their binding domain, FKBP12 permits specific activation of the recombinant receptor in vivo without the induction of non-specific side effects through endogenous FKBP12. In addition, the synthetic ligands are resistant to protease degradation, making them more efficient at activating receptors in vivo than most delivered protein agents.

In specific embodiments of the present invention, rapamycin analogs crosslink endogenous FKBP12 with a 90 amino acid domain from FRAP/mTOR, called FRB (FRAP rapamycin binding domain, residues 2025-2113). Thus, in specific embodiments of the present invention, activation of iAkt is based on ligand-dependent recruitment of chimeric Akt (first chimeric protein) to a membrane-bound myristoylated “docking protein” (second chimeric protein).

The ligands used in the present invention are capable of binding to two or more of the ligand-binding domains. One skilled in the art realizes that the chimeric proteins may be able to bind to more than one ligand when they contain more than one ligand-binding domain. The ligand is typically a non-protein or a chemical. Exemplary ligands include, but are not limited to dimeric FK506 (e.g., FK1012), AP1903, rapamycin or a derivative thereof.

B. Selectable Markers

In certain embodiments of the invention, the expression constructs of the present invention contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) are employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, μgal or chloramphenicol acetyltransferase (CAT).

C. Control Regions

1. Promoters

The particular promoter employed to control the expression of a polynucleotide sequence of interest is not believed to be important, so long as it is capable of directing the expression of the polynucleotide in the targeted cell. Thus, where a human cell is targeted, it is preferable to position the polynucleotide sequence-coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, elongation factor 1-alpha (EF-1α), rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene products are toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Offm system would be preferable so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Similarly tissue specific promoters are used to effect transcription in specific tissues or cells so as to reduce potential toxicity or undesirable effects to non-targeted tissues. For example, promoters such as the alpha myosin heavy chain (αMHC) promoter, directing expression to cardiac myocytes.

In certain indications, it is desirable to activate transcription at specific times after administration of the gene therapy vector. This is done with such promoters as those that are hormone or cytokine regulatable. Cytokine and inflammatory protein responsive promoters that can be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antityrpsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and gluccocorticoid inducible), Stromelysin (inducible by phorbol ester, interleukin-1 and EGF), alpha-2 macroglobulin and alpha-1 antichymotrypsin.

It is envisioned that any of the above promoters alone or in combination with another can be useful according to the present invention depending on the action desired. In addition, this list of promoters should not be construed to be exhaustive or limiting, those of skill in the art will know of other promoters that are used in conjunction with the promoters and methods disclosed herein.

2. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Any promoter/enhancer combination (as per the Eukaryotic Promoter Data Base EPDB) can be used to drive expression of the gene. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence is employed such as human or bovine growth hormone and SV40 polyadenylation signals. Also contemplated as an element of the expression cassette is a terminator. These elements can serve to enhance message levels and to minimize read through from the cassette into other sequences.

4. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be in-frame with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic messages. IRES elements are able to bypass the ribosome-scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

II. Methods of Gene Transfer

In order to mediate the effect of the transgene expression in a cell, it will be necessary to transfer the expression constructs of the present invention into a cell. Such transfer may employ viral or non-viral methods of gene transfer. This section provides a discussion of methods and compositions of gene transfer.

A. Non-viral Transfer

Several non-viral methods for the transfer of expression constructs into cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

In a specific embodiment of the present invention, the expression construct is complexed to a cationic polymer. Cationic polymers, which are water-soluble complexes, are well known in the art and have been utilized as a delivery system for DNA plasmids. This strategy employs the use of a soluble system, which will convey the DNA into the cells via a receptor-mediated endocytosis (Wu & Wu 1988). One skilled in the art realizes that the complexing nucleic acids with a cationic polymer will help neutralize the negative charge of the nucleic acid allowing increased endocytic uptake. Exemplary cationic polymers include, but are not limited to, polylysine, polyethyleneimine, polyhistidine, protamine, polyvinylamines, polyvinylpyridine, polymethacrylates, and polyornithine.

In a particular embodiment of the invention, the expression construct is entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection. Also included are various commercial approaches involving “lipofection” technology.

In certain embodiments of the invention, the liposome is complexed with a hemagglutinating virus (HVJ). This has been shown to facilitate fusion with the cell membrane and promote cell entry of liposome-encapsulated DNA (Kaneda et al., 1989). In other embodiments, the liposome is complexed or employed in conjunction with nuclear nonhistone chromosomal proteins (HMG-1) (Kato et al., 1991). In yet further embodiments, the liposome is complexed or employed in conjunction with both HVJ and HMG-1. In that such expression constructs have been successfully employed in transfer and expression of nucleic acid in vitro and in vivo, then they are applicable for the present invention.

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also is specifically delivered into a cell type such as prostate, epithelial or tumor cells, by any number of receptor-ligand systems with or without liposomes. For example, the human prostate-specific antigen (Watt et al., 1986) is used as the receptor for mediated delivery of a nucleic acid in prostate tissue.

In another embodiment of the invention, the expression construct may simply consist of naked recombinant DNA or plasmids. Transfer of the construct is performed by any of the methods mentioned above which physically or chemically permeabilize the cell membrane. This is applicable particularly for transfer in vitro, however, it is applied for in vivo use as well. Dubensky et al., (1984) successfully injected polyomavirus DNA in the form of CaPO4 precipitates into liver and spleen of adult and newborn mice demonstrating active viral replication and acute infection. Benvenisty and Neshif (1986) also demonstrated that direct intraperitoneal injection of CaPO4 precipitated plasmids results in expression of the transfected genes. It is envisioned that DNA encoding a CAM also is transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

B. Viral Vector-Mediated Transfer

In certain embodiments, transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

2. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and ψ components is constructed (Mann et al., 1983). When a recombinant plasmid containing a human cDNA, together with the retroviral LTR and ψ sequences is introduced into this cell line (by calcium phosphate precipitation for example), the ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

3. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1995; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

4. Other Viral Vectors

Other viral vectors are employed as expression constructs in the present invention. Vectors derived from viruses such as vaccinia virus (Ridgeway, 1988; Baichwal and Sugden, 1986; Coupar et al., 1988) canary pox virus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

III. Methods for Screening Active Compounds

The present invention also contemplates the use of the expression constructs and active fragments, and the corresponding nucleic acids encoding thereof, in the screening of compounds that can down-regulate, dephosphorylate or indirectly phosphorylate cells containing constitutively active Akt alleles. These assays may make use of a variety of different formats and may depend on the kind of “activity” for which the screen is being conducted.

In certain embodiments, it is provided a method of screening compounds to identify a modulator of Akt comprising the steps of: providing a cell expressing iAkt; contacting the cell with a candidate compound; admixing rapamycin analogs to induce activation of Akt; measuring the level of expression or activity of Akt; and comparing the Akt expression or activity in the presence of the candidate modulator with the expression or activity of Akt in the absence of the candidate modulator; wherein a difference in the expression or activity of Akt in the presence of the candidate modulator, as compared with the expression or activity of Akt in the absence of the candidate modulator, identifies the candidate modulator as a modulator of Akt expression or activity.

As used herein the term “candidate substance” refers to any molecule that may potentially inhibit or enhance Akt expression or activity. The candidate substance may be a protein or fragment thereof, a small molecule, or even a nucleic acid molecule. It may prove to be the case that the most useful pharmacological compounds will be compounds that are structurally related to Akt nucleic acid sequence and/or amino acid sequence. Using lead compounds to help develop improved compounds is know as “rational drug design” and includes not only comparisons with know inhibitors and activators, but predictions relating to the structure of target molecules.

A. In vitro Assays

In one embodiment, the invention is to be applied for the screening of compounds that bind to the Akt nucleic acid, polypeptide or fragment thereof. The nucleic acid, polypeptide or fragment may be either free in solution, fixed to a support, expressed in or on the surface of a cell. Either the nucleic acid, polypeptide or the compound may be labeled, thereby permitting determining of binding.

In another embodiment, the assay may measure the inhibition of binding of Akt to a natural or artificial substrate or binding partner. Competitive binding assays can be performed in which one of the agents (Akt, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with Akt and washed. Bound polypeptide is detected by various methods.

Purified Akt can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the Akt active region to a solid phase.

Various cell lines containing the Akt expression construct of the present invention can be used to study various functional attributes of Akt and how a candidate compound affects these attributes. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of Akt, or related pathways, may be explored.

B. In vivo Assays

The present invention also encompasses the use of various animal models. Thus, any identity seen between human and other animal Akt molecules provides an excellent opportunity to examine the function of Akt expression, Akt activity and/or compositions or compounds that interact with Akt in a whole animal system. By developing or isolating cells lines that express inducible Akt, compounds and/or compositions can be studied that modulate Akt activity. Yet further, since Akt is often upregulated in various tumors and viral Akt is an oncogene, it is envisioned that cells lines and/or animals that express inducible Akt of the present invention in a tissue or non-tissue specific manner can lead to neoplastic models.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, cell survival, decreased apoptosis, decrease in apoptotic proteins, decrease in membrane blebbing, retention of cell integrity, decrease in cellular and cytoplasmic shrinkage, decrease in chromosome fragmentation and condensation, or a decrease in endonuclease activation.

C. Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs, which are more active or stable than the natural molecules, which have different susceptibility to alteration or which may affect the function of various other molecules. In one approach, one would generate a three-dimensional structure for Akt or a fragment thereof. This could be accomplished by x-ray crystallography, computer modeling or by a combination of both approaches. An alternative approach, “alanine scan,” involves the random replacement of residues throughout molecule with alanine, and the resulting affect on function determined.

It also is possible to isolate a Akt-specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved Akt activity or which act as stimulators, inhibitors, agonists, antagonists of Akt. By virtue of the availability of cloned Akt gene sequences, sufficient amounts of Akt can be produced to perform crystallographic studies. In addition, knowledge of the polypeptide sequences permits computer employed predictions of structure-function relationships.

D. Transgenic Animals

In one embodiment of the invention, transgenic animals are produced which contain a functional transgene encoding an inducible functional Akt polypeptide or variants thereof. Transgenic animals expressing inducible Akt transgenes, recombinant cell lines derived from such animals and transgenic embryos may be useful in methods for screening for and identifying agents that induce or repress function of Akt. Transgenic animals of the present invention also can be used as models for studying disease states. Still further, transgenic animals can be used to create conditional or inducible neoplasm or hyperplasia animal models, which are used to study the mechansims of the disease or to determine substrates that may be used to develop drugs or as a mechanism to test candidate substances for their potential in inhibiting or prohibinting neoplasm or hyperplasia growth or development.

In one embodiment of the invention, an inducible Akt transgene is introduced into a non-human host to produce a transgenic animal expressing a human or murine Akt gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994)).

Typically, a Akt gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

As noted above, transgenic animals and cell lines derived from such animals may find use in certain testing experiments. In this regard, transgenic animals and cell lines capable of expressing an inducible Akt may be exposed to test substances. These test substances can be screened for the ability to enhance Akt expression and or function or impair the expression or function of Akt.

IV. Methods for Treating

The present invention also contemplates a method of modulating apoptosis in a cell susceptible to apoptosis comprising the steps of: administering to the cell an inducible Akt expression vector of the present invention, and modulating apoptosis with ligands comprising rapamycin analogs. One skilled in the art is fully aware that activation of the Akt molecule of the present invention relies upon CID to induce its activity. Thus, it is understood that in a second expression vector encoding a second chimeric protein a ligand-binding domain fused to a membrane-targeting region is also necessary for induction of expression. One skilled in the art is aware that the polynucleotide sequences for the second chimeric protein can be included in the Akt expression vector in tandem under control of a separate promoter or separated by an internal ribosome entry sequence, which results in a bicistronic construct. The previous discussion of the expression vectors of the present invention is thus incorporated herein.

It is envisioned that the Akt molecule is administered to a stem cell to increase the life-span of the stem cell. Yet further, the Akt molecule is administered to a transplant cell to enhance or regulate the survival of gene-modified transplant cells.

It is also envisioned that the inducible Akt expression vector or fragment thereof is administered to a cell, tissue or animal to modulate hypoxia-induced apoptosis or tissue damage following ischemia-reperfusion. In the present invention, animal includes, but is not limited to mammals, such as human, non-human primate, cow, horse, pig, sheep, goat, dog, cat, or rodent.

A host cell can, and has been, used as a recipient for vectors, for example the Akt expression vector of the present invention. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials. In certain embodiments, a cell may comprise, but is not limited to, at least one skin, bone, neuron, axon, cartilage, blood vessel, cornea, muscle, facia, brain, prostate, breast, endometrium, lung, pancreas, small intestine, blood, liver, testes, ovaries, cervix, colon, skin, stomach, esophagus, spleen, lymph node, bone marrow, kidney, peripheral blood, embryonic or ascite cell, and all cancers thereof. An appropriate host can be determined by one of skill in the art based on the vector backbone and the desired result. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

In certain embodiments, a tissue may comprise, but is not limited to, adipocytes, alveolar, ameloblasts, axon, basal cells, blood (e.g., lymphocytes), blood vessel, bone, bone marrow, peripheral stem cells, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, or ascite tissue.

In certain embodiments, the cell or tissue may be comprised in at least one organism. In certain embodiments, the organism may be, but is not limited to, an eubacteria, an archaea, an eukaryote or a virus. Specifically the organism is an eukaryote (e.g., a protist, a plant, a fungi, an animal). In particular embodiments, the eukaryote may be, but is not limited to, a microsporidia, a diplomonad, an oxymonad, a retortamonad, a parabasalid, a pelobiont, an entamoebae or a mitochondrial eukaryote (e.g., an animal, a plant, a fungi, a stramenopiles).

In certain embodiments, the mitochondrial eukaryote may be, but is not limited to, a metazoa (e.g., an animal), a myxozoa, a choanoflagellate, a fungi (e.g., a mushroom, a mold, a yeast, a chytrid), a green plant (e.g., a green algae, a land plant), a cryptomonad, an ancyromona, plasmodiophorid, a rhodophyta, a centrohelid heliozoa, a cyanophorid, an alveolate (e.g., a dinoflagellate, a sporozoan, a ciliate), a stramenopile (e.g., a brown algae, a diatoms, an oomycete, a chrysophyte), an acantharea, a vampyrellid, a thaumatomonad, a telonema, a sticholonche, a spongomonad, a ramicristate, a pseudospora, a pseudodendromonad, a phalansterium, a phaeodarean radiolaria, a paramyxea, a luffisphaera, a leucodictyon, a kathablepharid, a histiona, a haptophyte, an ebriid, a discocelis, a diphylleia, a eesmothoracid, a cryothecomona, a copromyxid, a chlorarachnion, a cercomonad, a caecitellus, an apusomonad, an actinophryid or an acanthamoebae. A chordata (e.g., a vertebrate) is preferred.

In particular facets the vertebrate may be a terrestrial vertebrate (e.g., a frog, a salamander, a caecilian, a reptile, a mammal, a bird) or a non-terrestrial vertebrate (e.g., a sharks, a ray, a sawfish, a chimera, a ray-finned fish, a lobe-finned fish). In additional facets, the mammal may be a monotremata (e.g., a platypus, an echidna), a multituberculata, a marsupialia (e.g., an opossum, a kangaroo), a palaeoryctoids or an eutheria (e.g., a placental mammal).

In certain embodiments wherein the Akt molecule is administered to an apoptotic cell, the administration of the Akt molecule can be either acute or prophylactic. Such acute and/or prophylactic administration of the Akt molecule is contemplated when the cell is part of a tissue or an organ to be transplanted or implanted. It is envisioned that administration of the Akt molecule allows for longer term survival of the cells of the transplanted and/or implanted tissue or organ under the adverse conditions the tissue or organ is subjected to during such procedure, e.g., ischemia, lower temperature, reperfusion, etc., therefore improving the tissue or organ's viability and/or acceptance by the recipient organism.

In certain embodiments, the present invention envisions treating myocardial infarction comprising the step of: administering to a subject in need of such treatment an inducible Akt molecule in an amount effective to reduce cardiac tissue necrosis in the subject. In addition to myocardial infarction other diseases associated with cardiomyocyte apoptotic cell-death (e.g., myocardial infarction, ischemia-reperfusion injury, dilated cardiomyopathy, conductive system disorders) can be treated using the Akt molecule of the present invention.

When the Akt molecule is used therapeutically, the molecule is administered in therapeutically effective amounts. In general, a therapeutically effective amount means that amount necessary to delay the onset of, inhibit the progression of, or halt altogether the particular condition being treated. A therapeutically effective amount will vary with the subject's age, condition, and sex, as well as the nature and extent of the disease in the subject, all of which can be determined by one of ordinary skill in the art. Thus, the therapeutically effective amount of the Akt molecule is that amount effective to inhibit increased apoptotic cell-death of a cell and can be determined using, for example, standard tests, known in the art. Standard tests include, but are not limited to TUNEL staining, and the appearance of condensed chromatin and other morphological features characteristic of apoptosis in electron micrographs.

It is important to recognize that the Akt molecule of the present invention is a “survival switch”. Thus, the Akt molecule of the present invention can be administered as a nucleic acid molecule and/or chimeric protein to enhance the survival of cells and/or tissues in vitro or in vivo.

In certain embodiments, a disease is treated by screening for a modulator of Akt and then administering to a subject or animal suffering from the disease the modulator of Akt. It is contemplated that the disease is a hyperproliferative disease and may be treated by administering to a subject an effective amount of an Akt modulator. The subject is preferably a mammal and more preferably a human.

In the present invention, a hyperproliferative disease is further defined as cancer. In still further embodiments, the cancer is melanoma, non-small cell lung, small-cell lung, lung, leukemia, hepatocarcinoma, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

In other embodiments, the hyperproliferative disease is rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

A. Genetic Based Therapies

Specifically, the present inventors intend to provide, to a cell, an expression construct capable of providing Akt to that cell and activate Akt. The lengthy discussion of expression vectors and the genetic elements employed therein is incorporated into this section by reference. Particularly preferred expression vectors are viral vectors such as adenovirus, adeno-associated virus, herpes virus, vaccinia virus and retrovirus. Also preferred is lysosomal-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

B. Protein Therapy

Another therapy approach is the provision, to a subject, of Akt polypeptide, active fragments, synthetic peptides, mimetics or other analogs thereof. The protein may be produced by recombinant expression means. Formulations would be selected based on the route of administration and purpose including, but not limited to, liposomal formulations and classic pharmaceutical preparations.

C. Combined Therapy

In order to increase the effectiveness of the Akt molecule of the present invention, it is desirable to combine these compositions with an additional agent. For example, known suicide genes, anti-apoptotic genes or proteins, or growth factors that are known to act cooperatively, additively or synergistically with Akt can be added to the invention to inhibit or reduce apoptotic cell-death.

The Akt molecules may precede, be co-current with and/or follow the other agent(s) by intervals ranging from minutes to weeks. In embodiments where the Akt molecule modulator, and other agent(s) are applied separately to a cell, tissue or organism, one would generally ensure that a significant period of time did not expire between the time of each delivery, such that the Akt molecule and agent(s) would still be able to exert an advantageously combined effect on the cell, tissue or organism.

Various combination regimens of the Akt molecules and one or more agents are employed. One of skill in the art is aware that the Akt molecules and agents can be administered in any order or combination.

Administration of the composition Akt molecules to a cell, tissue or organism may follow general protocols for the administration of agents, taking into account the toxicity, if any. It is expected that the treatment cycles would be repeated as necessary. In particular embodiments, it is contemplated that various additional agents are applied in any combination with the present invention.

V. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Plasmid Construction

To generate F3-Akt, F3-DPH.Akt, F3-AktKM and variants, Akt and ΔPH. Akt were Pfu (Stratagene)-amplified from pCMV6-HA-Akt (Bellacosa et al., 1993) or pCMV6-HA-AktK179M (Datta et al., 1997) using SalI-linkered 5′ primers, mAkt5SPH (full-length): SEQ.ID.NO:6 (5′-agagcgacaacgacgtagccattgtgaaggag-3′) or mAkt5S (truncated ΔPH): SEQ.ID.NO:7 (5′-agagtcgacaccgccattcagactgtggcc-3′) and 3′ primer, mAkt3S: SEQ.ID.NO:8 (5′-agagtcgacggctgtgccactggctgagtag-3′). PCR products were subcloned into pCR-Blunt (Invitrogen) or pKSII+(Stratagene) and sequence verified, to createpSH5/mAkt, pSH5/mΔPH.Akt, and pKS/mAkt.KM. The 1440-bp full-length Akt and 1130-bp ΔPH.Akt fragments were removed with SalI and subcloned into XhoI/SalI-digested M-Fpk 3-E, or XhoI or SalI-digested S-Fₚₖ 3-E (MacCorkle et al., 1998), to create M-Akt (and Akt variants), Akt-F3 (and variants) and F3-Akt (and variants). All chimeric proteins contain the HA epitope (E), but the “E” is left off (along with “pk” subscripts) for simplicity.

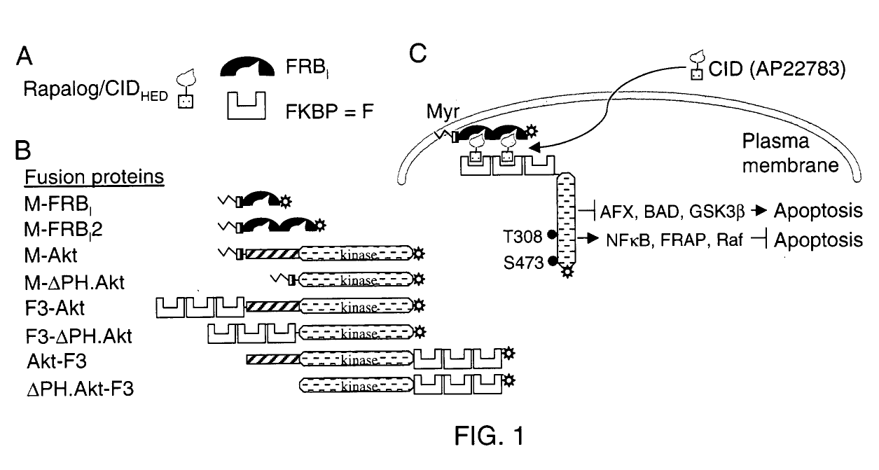

As shown in FIG. 1A, the heterodimeric rapalog/CID_{HED} can effect the crosslinking of FRB1 and FKPB12 (called F). In these experiments, the non-toxic variant of FKBP12, Fpk (FKBP12(G89P, I90K)), was used to eliminate background toxicity.

To generate myristoylated rapalogue-binding domains, the rapamycin binding domain (FRB) from human FRAP (res. 2025-2113; T2098L) was PfuI-amplified from FRAP*-AD (Pollock et al., 2000) using primers SEQ.ID.NO:9 (5′-cgatctcgaggagatgtggcatgaaggcctgg-3′) and 3FRBS: SEQ.ID.NO:10 (5′-cgatgtcgacctttgagattcgtcggaacacatg-3′) and subcloned into pCR-Blunt to produce pSH5/FRB₁. One or two copies of the XhoI/SalI FRB1 domain were subcloned into XhoI/SalI-digested M-Fₚₖ 3-E to create M-FRB₁ and M-FRB₁ 2. Also, the NF-κB-SEAP reporter plasmid was produced (Spencer, 1996). Thus, a constitutively active, myristoylated Akt (M-Akt) or M-ΔPH.Akt and kinase-dead mutant versions (i.e. Akt.K179M, named AktKM) of chimeric Akt constructs were developed (FIG. 1B).

To make the bicistronic iAkt constructs, two different internal ribosome entry sequence (IRES) elements from EMCV or poliovirus were used to link M-FRB₁2 and F3-ΔPH.AKT on the same transcript. The poliovirus 1RES sequence (IRESp) was PfuI-amplified from pTPOV-3816 (Lloyd et al., 1988) with primers, 5pIRES/Mn: SEQ.ID.NO:11 (5′-atacaattgccgcggttcgaattctgttttatactcccttcccgtaac-3′) and 3pIRES/Mun; SEQ.ID.NO: 12 (5′-tatcaattggtttaaacagcaaacagatagataatgagtctcac-3′). The resulting PCR products were subcloned into pCR-Blunt to create pSH5/IRESp-Mun. The 615-bp IRESp MunI fragment was ligated into EcoRI-digested pSH1/M-FRB₁2-E to create pSH1/M-FRB₁2-E-IRESp. Finally, the NotI/EcoRI F3-ΔPH.AKT fragment from pSH1/F3-ΔPH.AKT was blunt-ligated into the PmeI site to create pSH1/M-FRB₁ 2-E-IRESp-F3ΔPH.Akt, renamed as iAkt_{b}. The bicistronic vector iAktₐ utilizes the EMCV IRES and was made by a comparable strategy.

For establishing Jurkat.iAkt cell lines, the bicistronic NotI/MunI fragment from iAkt_{b} was subcloned into NotI/EcoRI-digested pBJ5-neo to create pBJ5-neo/iAkt_{b}.

To create inducible Akt (iAkt) molecules, three tandem FKBP domains (F3) were fused to the N- or C-termini of wild-type Akt or a variant. ΔPH.Akt), lacking the pleckstrin homology (PH) domain to reduce natural membrane association.

Thus, membrane recruitment of F3-modified Akt by rapalogs, such as AP22783 used in these experiments, leads to phosphorylation of Akt at T308 and S473, induction of Akt kinase activity, and phosphorylation of downstream effector molecules, leading to modulation of apoptotic signals.

### Example 2

### Cell Culture

293T human embryonic kidney cells (ATCC) and Jurkat (ATCC), Jurkat-TAg (Northrop et al., 1993) and Jurkat.iAkt were maintained in DMEM or RPMI-1640, respectively, containing 10% fetal bovine serum (FBS) and antibiotics. The Jurkat.iAkt line was derived by transfecting Jurkat cells with NdeI-linearized pBJ5-neo/iAkt_{b} plasmid followed by G418 (1 mg/ml) selection. Clones were screened by anti-HA immunoblotting.

### Example 3

### Electroporation and SEAP Assay

Jurkat-TAg cells in logarithmic-phase growth were electroporated (950 mF, 250 V; Gene Pulser II (BIO-RAD)) with expression plasmids and the NF-κB-SEAP reporter plasmid. After 24 hours, transfected cells were stimulated with sub-optimal levels of the phorbol ester PMA (5 ng/ml) along with log dilutions of the heterodimerizing CID, AP22783, and additional treatments. After an additional 24 hours, supernatants were assayed for SEAP activity (Spencer et al., 1993).

### Example 4

### Western Blots

293T cells seeded in 6-well plates were transiently transfected with 2 μg of different expression constructs in 6 μl FuGENE6 (Boehringer-Mannheim, Indianapolis, Ind.) in Opti-MEM-I medium for 24 hours followed by serum starvation for an additional 24 hours. Cells were then treated with different agents and harvested at different time points. After washing (2×) in ice-cold PBS, cell pellets were lysed in RIPA buffer containing protease inhibitors (CytoSignal, Irvine, Calif.). Equal amounts of protein from each sample were separated on 10% SDS-PAGE gels and transferred to PVDF membrane (Amersham Pharmacia Biotech, Piscataway, N.J.). Phospho-specific antibodies against Akt (T308 or S473 site) (Cell Signaling, Beverly, Mass.) were used for measuring Akt phosphorylation, and the signal was detected by AP-conjugated secondary antibodies (NEB, Beverly, Mass.) and CDP-Star chemiluminescence reagent (NEN life science, Boston, Mass.).

### Example 5

### Immunoprecipitation and in vitro Akt Kinase Assay

Jurkat.iAkt_{b} were serum starved for 24 hours followed by treatment with AP22783 or serum for 30 min. Cells were then lysed in a lysis buffer provided with the Akt Kinase Assay kit (Cell Signaling, Beverly, Mass.), and Fₚₖ3-ΔPH.AKT-E was immunoprecipitated with polyclonal anti-HA antibody. Antibody-antigen complexes were washed three times in lysis buffer and once in kinase buffer. In vitro kinase assays for Akt were performed using a GSK3α/β “crosstide”. The extent of crosstide phosphorylation was determined by anti-GSKα/β immunoblotting according to the manufacturer's protocol.

### Example 6

Apoptosis and Flow Cytometry

Jurkat.iAkt were serum starved for 24 hours followed by pre-treatment with AP22783 in 0, 2 or 10% FBS for 40 min. After incubation with apoptosis-inducing stimuli for the periods indicated, cells were harvested and washed twice in ice-cold PBS and fixed in 70% ethanol. Cells were stained in 50 μg/ml propidium iodide and 100 μg/mlRNase A for 30 min at 37° C., and hypodiploid cells were quantitated by flow cytometry using a Beckman-Coulter EPICS XL-MCL.

For determination of caspase-3 activation and PARP cleavage after staurosporine treatment, cell pellets were lysed in Laemmli sample buffer containing 5% (v/v) β-mercaptoethanol (Bio-Rad, Hercules, Calif.), and equal amounts of protein were separated on 6 (for PARP) or 12% (for caspase3) SDS-PAGE followed by immunoblotting with anti-caspase-3 and anti-PARP antibodies.

### Example 7

### Membrane-Targeting of PH Domain-Less Akt Leads to Rapalogdose-Dependent Activation of NF-κB

Two key requirements for efficient synthetic regulation of a biological event are highly specific conditional dependency and low background. NF-κB induction is a major target of Akt following growth factor signaling, and multiple reports show that a constitutively active myristoylated Akt (M-Akt) can enhance protein kinase C (PKC)-mediated NF-κB induction by either phosphorylation of IKKα, the activation domain of p65/RelA, or both. Therefore, in order to optimize iAkt, an NF-κB-responsive secreted alkaline phosphatase (SEAP) reporter plasmid was used as an assay for Akt activation.

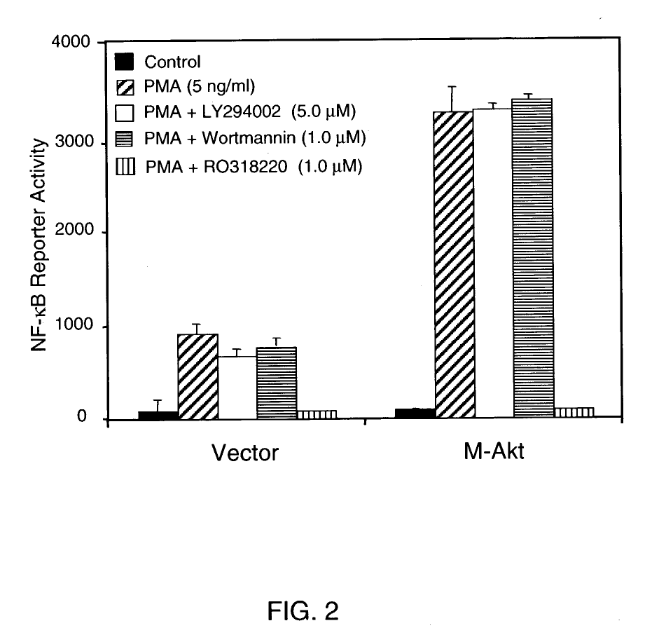

Briefly, the human T cell line, Jurkat-TAg, was cotransfected with reporter plasmid, NF-κB/SEAP, along with constitutively active M-Akt expression vector or empty control vector. Twenty-four hours after transfection, cells were divided into aliquots that were stimulated with sub-optimal levels (5 ng/ml) of the phorbol ester, PMA, or were untreated. After an additional 24 hours, SEAP activity was measured. Although Akt activity alone was insufficient to induce measurable NF-κB activity, M-Akt expression potentiated (by 3-4 fold) PKC-induced NF-κB activity, consistent with multiple reports. Furthermore, inhibition of PI3K by LY294002 (5 μM) or wortmannin (1 μM) did not prevent NF-κB activation by M-Akt plus PKC, although inhibition of “typical” PKC isoforms with R0318220 (1 μM) led to complete inhibition of NF-κB as expected (FIG. 2).

Since the constitutively active Akt (T308) kinase, PDK1, is primarily membrane-associated following growth factor stimulation, membrane recruitment of Akt via its PH domain is necessary for its activation. Furthermore, although the PH domain has been shown to suppress basal phosphorylation of T308 and Akt activation when not bound by its lipid ligand, PIP2, this initial phosphorylation should still require interaction with membrane-localized PDK1.

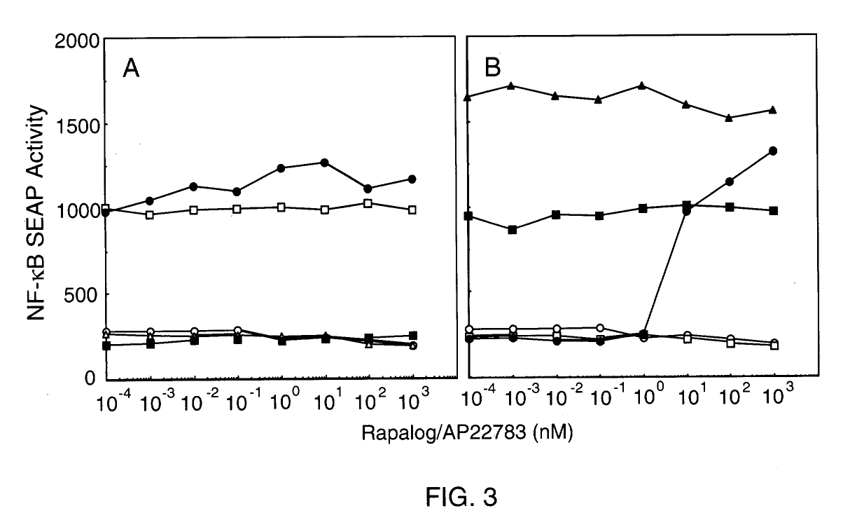

Basal Akt activity and activation following membrane recruitment of full length and truncated Akt, lacking the PH domain, was compared using a NF-κB reporter assay. Both full length and ΔPH.Akt were fused to a tandem trimer of the CID-binding domain, F3, at both the amino and carboxyl termini. As before Jurkat-TAg cells were cotransfected with reporter plasmid NF-κB/SEAP along with the membrane docking molecule, M-FRB₁2, alone, various F3-Akt chimeras, alone, or both together. Twenty-four hours after transfection, cells were stimulated with 5 ng/ml PMA along with log dilutions of heterodimerizing CID, AP22783. After additional 24 hr incubation, SEAP activity was assayed. As shown in FIG. 3A, wild type Akt showed significant CID-independent NF-κB induction that was only slightly increased by crosslinking to the membrane, via M-FRB12. This was true regardless of whether F3 was fused to the N- or C-terminus of Akt. As expected, membrane recruitment or overexpression of kinase-deficient Akt.KM (K179M) had no detectable effect on NF-κB induction over PMA alone. Thus, membrane recruitment of full-length Akt only slightly increases its activity due to the high basal activity from its overexpression. In contrast, membrane recruitment of F3-ΔPH.Akt showed a very clear CID-dependent induction of NF-κB with undetectable CID-independent activity (FIG. 3B). Moreover, myristoylated MΔPH.Akt was more active than M-Akt in augmenting NF-κB activation, consistent with an inhibitory function for the PH domain. Again, M-FRB₁ 2 alone or recruitment of kinase dead F3-ΔPH.AktKM did not influence NF-κB induction (FIG. 3B).

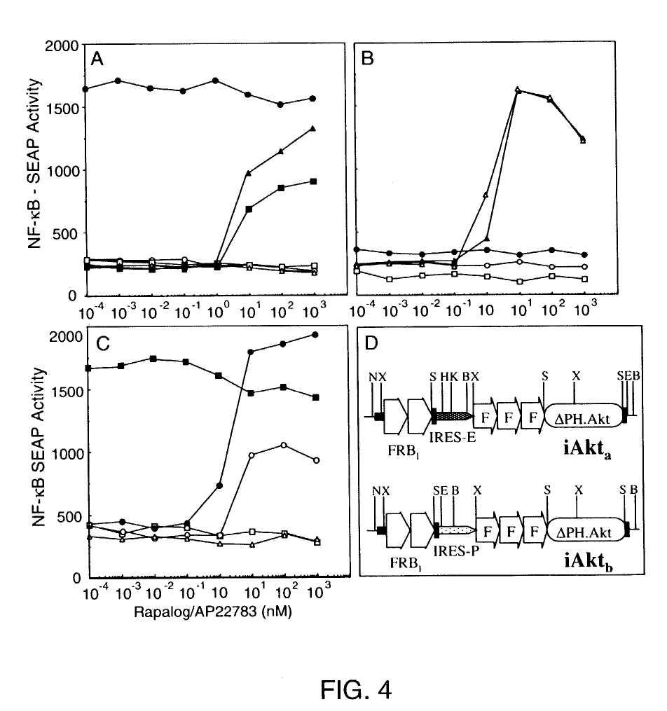

These results indicated that the chimeric F3-ΔPH.Akt allele is strongly CID-inducible with very low basal activity. Also, these results are consistent with previous reports that the PH domain of Akt kinase is responsible for its translocation to the plasma membrane and also has an inhibitory function. Since most applications of CID technology have been based partly, at least, on empirically designed inducible chimeric proteins, CID-mediated targeting or crosslinking might not always faithfully reflect physiological signaling. Further, CID-binding domains, like FKBP12, could potentially sterically hinder an essential target protein domain(s). Therefore, ΔPH.Akt with F3 fused to both termini of ΔPH.Akt was tested. As shown in FIG. 4A, the N-terminal fusion chimera, F3-ΔPH.Akt, potentiated NF-κB transactivation somewhat better than the C-terminal chimera, ΔPH.Akt-F3. Since both molecules were expressed at similar levels, membrane recruitment of F3-ΔPH.Akt may place Akt in a more favorable orientation for interacting withPDK1 or other interacting proteins. In either orientation, however, both iAkt versions were devoid of detectable basal NF-κB signaling.

Since, M-FRB₁ 2 could potentially recruit two chimeric Akt molecules simultaneously, it was determine if membrane recruitment of one Akt molecule was sufficient for optimal activation or whether oligomerization of multiple Akt molecules might enhance activation. Therefore, CID-mediated iAkt activity was compared when the membrane docking molecule, contained one or two FRB₁ domains (FRB₁ vs FRB₁ 2, respectively). As shown in FIG. 4B, there was no significant difference in NF-κB induction by iAkt whether one or two tandem FRB₁ domains were used for the docking site, indicating that forced Akt oligomerization is not a prerequisite for its activation.

### Example 8

### CID-Dependent Membrane-Targeting of Akt Kinase Results in Rapid Phosphorylation and Activation

Following membrane-targeting of endogenous Akt-1, phosphorylation at two highly conserved residues, T308 in the activation loop, and S473 in the inhibitory domain, occurs by PDK1 and PDK2, respectively.

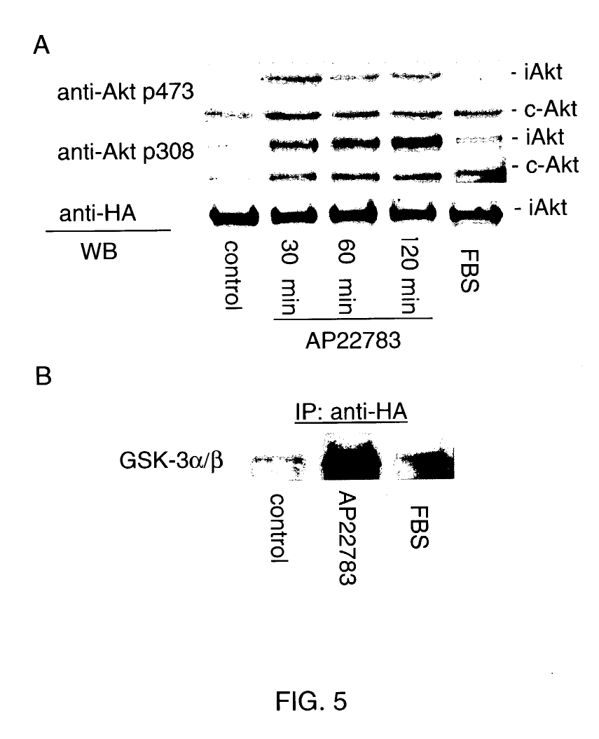

Briefly, 293T cells were transiently co-transfected with M-FRB₁ 2 and F3-ΔPH.Akt expression plasmids. After 24 hours, transfected cells were serum starved for another 24 hours and then treated with AP22783. As shown in FIG. 5A, AP22783 treatment greatly stimulated interaction with phospho-specific antibodies against Akt S473 and T308 sites as early as 30 min after drug addition. Although 120′ serum (10% FBS) treatment stimulated phosphorylation of the endogenous protein, serum did not stimulate phosphorylation of iAkt during this period. Activation of iAkt led to increased phosphorylation of endogenous Akt, particularly at S473.

Therefore, these results suggest that activation of iAkt led to partial activation of endogenous Akt. Thus, CID-mediated membrane-targeting of iAkt chimeras stimulated Akt phosphorylation.

For targeting tissues or cell lines with bigenic inducible proteins, multi-cistronic vectors were used to insure that two, or more, proteins were coexpressed. Two different bicistronic iAkt vectors were developed, using either the commonly used EMCV internal ribosome entry sequences (IRES), called iAktₐ, or iAkt_{b}, using the less characterized IRES from poliovirus. Following electroporation of bicistronic vectors into Jurkat cells and AP22783 stimulation, consistently higher NF-κB induction by iAkt_{b} was observed compared with iAktₐ (FIG. 4C). Therefore, iAkt_{b} was used to create variant Jurkat lines stably expressing both FRB₁ 2 and F3-ΔPH.Akt, called Jurkat.iAkt cells.

These results showed that the orientation of Akt and FKBP had only a minor influence on efficacy (FIG. 4A), membrane recruitment of iAkt without oligomerization was sufficient for activation (FIG. 4B) and the bicistronic vector, iAkt_{b}, containing M-FRB₁2 and F3-ΔPH.Akt separated by the poliovirus IRES, functioned more efficiently than iAktₐ which used the EMCV IRES (FIG. 4C).

To further measure the induction of Akt enzymatic activity, Jurkat.iAkt cells were serum starved for 24 hours and thereafter treated with AP22783 or serum for 30 min. Chimeric F3-ΔPH.Akt.-kinase was immunoprecipitated with the anti-HA antibody and Akt kinase activity was measured using an in vitro kinase assay that uses a GSK3α/β “crosstide” as a substrate. The level of phosphorylated GSK3 crosstides was determined by immunoblotting with phospho-specific antibody against GSK3-α21/β9. As shown in FIG. 5B, only AP22783 treatment, but not serum or mock treatment, was associated with GSK3 phosphorylation in this assay, indicating, as above, Akt activation after CID-mediated membrane-targeting.

### Example 9

### CID-Mediated Membrane-Targeting of iAkt Results in PI3K-Independent Activation

For maximum utility, an ideal CID-inducible protein would respond only to CID, but not to environmental signals, such as growth factors. Since endogenous Akt is a major effector molecule of PI3K signaling, inhibition of PI3K leads to inhibition of c-Akt. However, the activity of membrane targeted Akt, such as M-Akt, is largely PI3K independent, presumably because basal levels of membrane-associated PDK1 are sufficient for Akt phosphorylation.

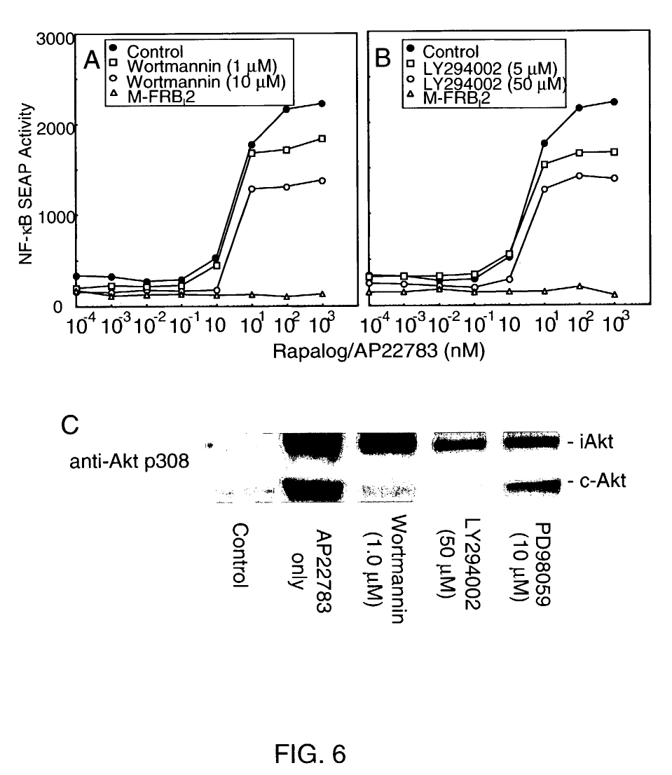

Next, two different inhibitors were used in the NF-κB reporter assay described above. Jurkat-TAg cells were cotransfected with reporter plasmid, NF-κB/SEAP, along with the bicistronic plasmid iAkt_{b}. After 24 hours, cells were pretreated with two different concentrations of either wortmannin (1 μM and 10 μM) or LY294002 (5 μM and 50 μM) for 40 min, and then cell aliquots were stimulated with 5 ng/ml PMA and log-dilutions of AP22783. SEAP activity was measured 24 hours later. As shown in FIG. 6A and FIG. 6B, the inhibitors at either concentration did not prevent NF-κB induction by iAkt, although maximal NF-κB activation was moderately effected.

Jurkat.iAkt cells were serum starved for 24 hours followed by 30 min pretreatment with PI3K inhibitors, wortmannin and LY294002, or MEK inhibitor, PD98059, as a control. After inhibitor pretreatment, AP22783 was added to the media for another 30 min to mobilize membrane recruitment of iAkt. As shown in FIG. 6C, addition of either PI3K inhibitor significantly blocked endogenous Akt phosphorylation at T308, but had a much smaller, if any (for 1 μM Wortmannin), effect on iAkt phosphorylation. The MAPK signaling inhibitor, PD98059, had no discernable effect on either endogenous or iAkt. These results, together with the NF-κB/SEAP assay (FIGS. 6A, 6B), indicated that CID-mediated iAkt activation was primarily independent of environmental signaling.

### Example 11

### CID-Mediated Activation of iAkt Leads to Apoptosis Resistance Following Multiple Pro-Apoptotic Signals

When overexpressed, wild type or constitutively active Akt has been demonstrated to protect cells from a variety of apoptotic stimuli, including treatment with DNA-damaging agents, PI3K inhibitors, Fas-crosslinking, UV (or γ) irradiation, c-myc overexpression, growth factor withdrawal, TGFβ treatment, matrix detachment, or cell cycle perturbation.

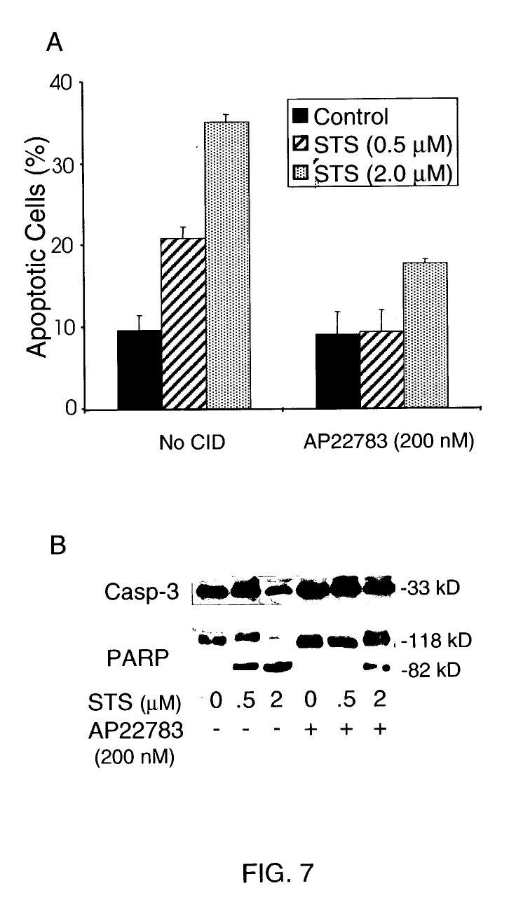

Jurkat.iAkt cells were given various apoptotic insults in the presence or absence of rapalogs. Initially, the protective effects of iAkt were studied on staurosporine (STS)-induced apoptosis. Although staurosporine is a potent inhibitor (IC50˜3 nM) of many PKC family members, it can also inhibit other S/T and tyrosine kinases at higher concentration. Therefore, the exact mechanism of triggering apoptosis is likely to be complex. Following serum starvation for 24 hours, Jurkat.iAkt cells were treated with 0, 0.5, or 2.0 μM STS with or without AP22783 (400 nM) for 6 hours in the absence of serum. Cell death was monitored using propidium iodide (PI) staining and flow cytometry (FCM) by quantitation of subdiploid cells. As expected, STS induced cell death in a dose-response manner (FIG. 7A). At low-dose STS treatment, 19.4% of cells were apoptotic after 6 hours, but this toxicity was fully blocked by AP22783 treatment. Further, although high-doses of STS treatment triggered greater apoptosis, iAkt activation was able to prevent, or delay, apoptosis for the majority (36% to 17% without background correction) of 2 μM STS-treated cells.

Next, caspase-3 activation and PARP substrate cleavage were measured by immunoblotting. As shown in FIG. 7B, STS treatment at low- and high-dose resulted in dramatic PARP cleavage and significant reduction of procaspase-3 (evidence for procaspase-3 activation) at high-dose. However, AP22783 addition blocked STS-induced caspase-3 activation and low-dose STS-induced PARP cleavage, and also reduced high-dose STS-induced PARP cleavage. Thus, activation of iAkt blocked or, at minimum, delayed apoptosis by the potent apoptosis inducer, STS.

As shown in FIG. 6, CID-mediated activation of chimeric ΔPH.Akt and induction of NF-κB were largely independent of PI3K.

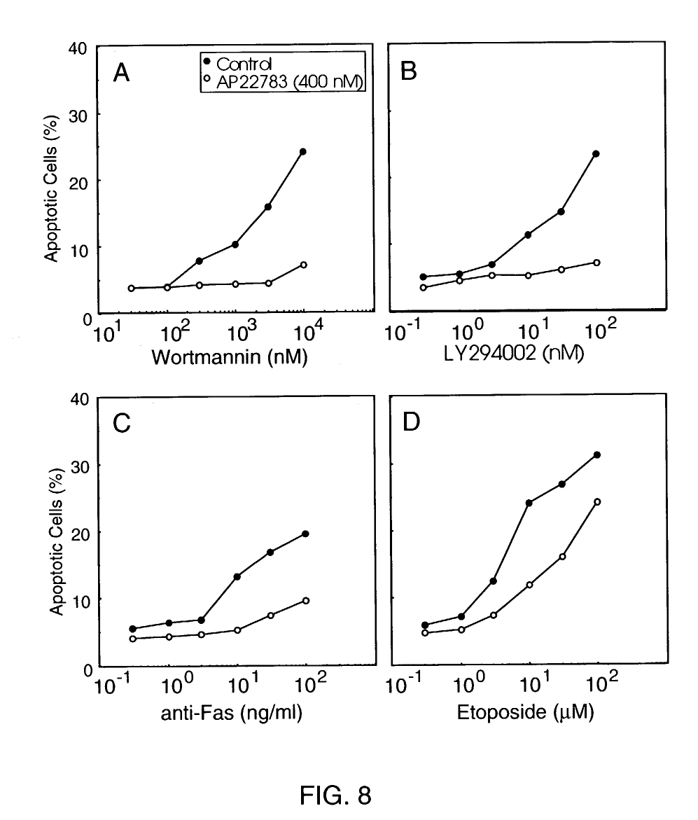

The effects of AP22783 treatment following PI3K inhibition of Jurkat.iAkt cell were studied. Serum-starved Jurkat.iAkt cells were treated with half-log dilutions of wortmannin (0.03-10 μM) or LY294002 (0.3-100 μM) with or without AP22783 (400 nM) in low (2%) or high (10%) FBS-containing medium for 9 hours (FIGS. 8A, 8B). In the presence of either low or high levels of FBS, the PI3K inhibitors induced increasing cell death in a very clear dose-dependent manner. In the presence of AP22783, however, cell death was almost totally blocked regardless of the FBS concentration. These results further demonstrated that CID-mediated Akt activation mitigated PI3K inhibition-induced cell death.

It is well established that oligomerization of the Fas receptor can result in rapid formation of the cytoplasmic death-inducing signaling complex (DISC) and activation of a caspase cascade that leads to apoptosis. Recent data has revealed that activation of the PI3K-Akt pathway can protect cells from Fas-mediated death. Therefore, the effect of CID-mediated Akt activation was studied on Fas-induced apoptosis in Jurkat.iAkt cells.

After 24 hours serum starvation, cells were treated with half-log dilutions (0.3-100 ng/ml) of the anti-Fas antibody, CH-11, in the presence or absence of AP22783 (400 nM) inlow (2%) or high (10%) FBS for 6 hours, as above. As shown in FIG. 8C, Fas receptor engagement-induced by CH-11 resulted in apoptosis in a dose-dependent fashion in both low and high FBS levels. However, regardless of the FBS concentration, AP22783 treatment rescued cells from CH-11-triggered apoptosis, indicating that the synthetic activation of iAkt was also able to partially protect cells from the deleterious effects of Fas signaling.

Finally, the anti-apoptotic effects of iAkt on protection from direct DNA damaging agents were examined. The anti-tumor agent, etoposide (VP16), is widely used as a chemotherapeutic drug that works by inhibiting DNA topoisomerase II, which can induce apoptosis in a variety of cell types. Briefly, Jurkat.iAkt cells were serum-starved for 24 hours followed by treatment with half-log dilutions of etoposide (0.3-100 μM) with or without AP22783 (400 nM) for 12 hours. Again, the experiments were carried out in two different culture conditions with low or high FBS levels. As shown in FIG. 8D, AP22783 addition reduced etoposide-induced cell death efficiently in high FBS conditions. A lesser, but reproducible level of protection was seen under low FBS conditions. Although, the ability of iAkt to delay, or block, apoptosis following etoposide treatment was not as extensive as blocking apoptosis triggered by STS, PI3K inhibition, or Fas ligation, these results demonstrated the CID-activated Akt was a powerful “live switch” to prevent, or delay apoptosis by multiple stimuli.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Alessi D. R. et al., Curr Biol 1997, 7:261-269.

Alessi D. R. et al., Curr Biol 1997, 7:776-789.

Anderson K. E. et al., Curr Biol 1998, 8:684-691.

Andjelkovic M. et al., J Biol Chem 1997, 272:31515-31524.

Antonawich F. J. et al., ExpNeurol 1999, 156:130-137.

Balendran A. et al., Curr Biol 1999, 9:393-404.

Bellacosa A. et al., Oncogene 1993, 8:745-754.

Bellacosa A. et al., Oncogene 1998, 17:313-325.

Bellacosa, A et al., Science 1991, 254:274-277.

Bilbao G. et al., Transplant Proc 1999, 31:1012-1015.

Bilbao G. et al., Transplantation 1999, 67:775-783.

Bilbao G. et al., Ann Surg 1999, 230:185-193.

Blau, C. A. et al., Proc Natl Acad.Sci. USA 1997, 94:3076-3081.

Bordignon, C. et al., Hum. Gene Ther. 1995, 6:813-819.

Cantley L. C. et al., Proc NatlAcad Sci USA 1999, 96:4240-4245.

Chan, T. O. et al., Annu Rev Biochem 1999, 68:965-1014.

Chen J. et al., Proc.Natl.Acad.Sci U.S.A. 1995, 92:4947-4951.

Choi J. et al., Science 1996, 273:239-242.

Contreras J. L. et al., Surgery 2001, 130:166-174.

Crabtree G. R. and Schreiber S. L. Trends.Biochem.Sci. 1996, 21:418-422.

Cross D. A. et al., Nature 1995,378:785-789.

Datta S. R. et al., Cell 1997, 91:231-241.

Datta, S. R. et al., Genes Dev 1999, 13:2905-2927.

Fadok V A, Nature 2000, 405:85.

Filippa N. et al., Mol Cell Biol 1999,19:4989-5000.

Franke T. F., et al., Science 1997, 275:665-668.

Franke, T. F. et al., Cell 1995, 81:727-736.

Gossen M. et al., Science 1995, 268:1766-1769.

Hausler P. et al., Eur J Immunol 1998, 28:57-69.

Hemmings B. A. Science 1997, 275:628-630.

Ho, S. N. et al., Nature 1996, 382:822-826.

Holsinger, L. J. et al., Proc.Natl.Acad.Sci. USA 1995, 95:9810-9814.

Jackson P. et al., EMBO J. 1993, 12:2809-2819.

Kane L. P. et al., Curr Biol 1999, 9:601-604.

Kaufmann S. H. Biochim Biophys Acta 1998, 1400:195-211.

Kohn A. D. et al., J Biol Chem 1996, 271:21920-21926.

Kohn A. D. et al., J Biol Chem 1998,273:11937-11943.

Kroner C. et al., J Biol Chem 2000, 275:27790-27798.

Lander, E. S. Nature 2001,409:860-921.

Lawrence M. S. et al., J Cereb Blood Flow Metab 1997, 17:740-744.

Li J. et al., Cancer Res 1998, 58:5667-5672.

Liberles S. D. et al., Proc.Natl.Acad.Sci. U.S.A. 1997, 94:7825-7830.

Linnik M. D. et al., Stroke 1995, 26:1670-1674; discussion 1675.

Liu A. X. et al., Cancer Res 1998, 58:2973-2977.

Lloyd R. E. et al., J Virol 1988, 62:4216-4223.

Lu Y. et al., J Exp Med 2001, 193:545-549.

Luo, Z. et al., Nature 1996,383:181-185.

MacCorkle, R. A. et al., Proc Natl Acad Sci USA 1998, 95:3655-3660.

Madrid L. V. et al., J Biol Chem 2001, 276:18934-18940.

Madrid L. V. et al., Mol Cell Biol 2000, 20:1626-1638.

Matsui T. et al., Circulation 2001, 104:330-335.

Meier R. et al., Embo J 1998, 17:7294-7303.

Mirza A. M., et al., Cell Growth Differ 2000,11:279-292.

Nakatani K. et al., J Biol Chem 1999, 274:21528-21532.

Northrop J. P. et al., J.Biol Chem 1993, 268:2917-2923.

Picard D. Curr.Opin.Biotechnol. 1994, 5:511-515.

Pollock R. et al., Proc Natl Acad Sci USA 2000, 97:13221-13226.

Rivera, V. M. et al., Nat.Med. 1996, 2:1028-1032.

Rohn J. L. et al., Oncogene 1998, 17:2811-2818.

Samuels M. L. et al., Mol Cell Biol 1993, 13:6241-6252.

Secrist J P. et al., J Biol Chem 1990,265:20394-20400.

Shaw M. et al., Biochem J 1998, 336:241-246.

Shimazaki K. et al., Gene Ther 2000,7:1244-1249.

Spencer D. M. et al., Curr Biol 1996, 6:839-847.

Spencer D. M. et al., Proc.Natl.Acad.Sci. USA 1995, 92:9805-9809.

Spencer D. M. et al., Science 1993, 262:1019-1024.

Spencer D. M. TIG 1996, 12:181-187.

Stambolic V. et al., Cell 1998, 95:29-39.

Tamaoki T. et al., Biochem BiophysRes Commun 1986, 135:397-402.

Thomis D. C. et al., Blood 2001, 97:1249-1257.

Toker A. et al., J Biol Chem 2000, 275:8271-8274.

Wymann, M. P. et al., Biochim Biophys Acta 1998, 1436:127-150.

Yuan Z. Q. et al., Oncogene 2000, 19:2324-2330.

Zheng L. et al., J Immunol 2000, 164:4665-4671.

Zhu W. Z, et al., Proc Natl Acad Sci USA 2001, 98:1607-1612.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. An expression vector comprising an inducible chimeric protein, wherein said protein comprises a mutant Akt polypeptide fused to a ligand-binding domain.

2. The expression vector of claim 1, wherein said ligand-binding domain is a derivative of FKBP.

3. The expression vector of claim 2, wherein the FKBP ligand-binding domain is FKBP506-Binding Protein.

4. The expression vector of claim 1 further comprising more than one ligand-binding domain.

5. The expression vector of claim 1, wherein said mutant Akt lacks a pleckstrin homology domain.

6. A host cell transformed with the expression vector of claim 1.

7. A fusion protein comprising a mutant Akt sequence and at least one ligand-binding domain.

8. The fusion protein of claim 7, wherein said ligand-binding domain is a derivative of FKBP.

9. The fusion protein of claim 7, wherein said mutant Akt lacks a pleckstrin homology domain.

10. A pharmaceutical composition comprising the expression vector of claim 1 and a pharmaceutically acceptable carrier.

11. A pharmaceutical composition comprising the fusion protein of claim 9 and a pharmaceutically acceptable carrier.

12. A method of modulating apoptosis comprising the steps of:
administering to a cell susceptible to apoptosis an expression vector encoding an inducible chimeric protein comprising a mutant Akt polypeptide fused to a ligand-binding domain;
administering to the cell a second expression vector encoding a second ligand-binding domain fused to a membrane-targeting region; and
modulating apoptosis by administering to the cell a chemical ligand, wherein the ligand results in activation of the mutant Akt.

13. The method of claim 12, wherein the first ligand-binding domain is a derivative of FKBP.

14. The method of claim 12, wherein the second ligand-binding domain is a rapamycin binding domain.

15. The method of claim 12, wherein the chemical ligand is a rapamycin analog.

16. The method of claim 12, wherein the membrane-targeting region is a myristoylated target sequence.

17. The method of claim 12 further comprising administering to the apoptotic cell an anti-apoptotic agent.

18. The method of claim 12 further comprising administering to the apoptotic cell a suicide gene.

19. A method of modulating apoptosis comprising the steps of:
administering to a cell susceptible to apoptosis an expression vector encoding an inducible chimeric protein comprising a mutant Akt polypeptide fused to a ligand-binding domain and a second chimeric protein comprising a ligand-binding domain fused to a membrane-targeting region; and
modulating apoptosis by administering to the cell a chemical ligand, wherein the ligand results in activation of the mutant Akt.

20. The method of claim 19, wherein said inducible chimeric protein and said second chimeric protein are separated by an internal ribosome entry sequence.

21. The method of claim 19, wherein said inducible chimeric protein and said second chimeric protein are under transcriptional control of two promoters.

22. A method of modulating apoptosis in a cell susceptible to apoptosis comprising the steps of administering the fusion protein of claim 7, administering a second fusion protein, wherein the second fusion protein comprises a second ligand-binding domain fused to a membrane-targeting region; and modulating apoptosis by administering to the cell a chemical ligand, wherein the chemical ligand results in activation of the mutant Akt.

23. A method of modulating hypoxia-induced apoptosis comprising the steps of:
administering to a cell suspected of hypoxia-induced apoptosis an expression vector encoding an inducible chimeric protein comprising a mutant Akt polypeptide fused to a ligand-binding domain;
administering to the cell a second expression vector encoding a second ligand-binding domain fused to a membrane-targeting region; and
modulating hypoxia-induced apoptosis by administering to the cell a chemical ligand, wherein the chemical ligand results in activation of the mutant Akt.

24. The method of claim 23, wherein said hypoxia-induced apoptosis is induced via ischemia.

25. A method of modulating a cell suspected of hypoxia-induced apoptosis comprising the steps of administering the fusion protein of claim 7, administering a second fusion protein, wherein the second fusion protein comprises a second ligand-binding domain fused to a membrane-targeting region; and modulating hypoxia-induced apoptosis by administering to the cell a chemical ligand, wherein the chemical ligand results in activation of the mutant Akt.

26. A method of modulating tissue damage following ischemia-reperfusion comprising the steps of:
administering to a tissue suspected of tissue damage an expression vector encoding an inducible chimeric protein comprising a mutant Akt polypeptide fused to a ligand-binding domain;
administering to the tissue a second expression vector encoding a second ligand-binding domain fused to a membrane-targeting region; and
modulating tissue damage by administering to the tissue a chemical ligand, wherein the ligand results in activation of the mutant Akt.

27. The method of claim 26, wherein said tissue is cardiac.

28. A method of modulating tissue damage following ischemia-reperfusion comprising the steps of administering to a tissue suspected of tissue damage the fusion protein of claim 7, administering to the tissue a second fusion protein, wherein the second fusion protein comprises a second ligand-binding domain fused to a membrane-targeting region; and modulating tissue damage by administering to the cell a chemical ligand, wherein the chemical ligand results in activation of the mutant Akt.

29. A method of treating myocardial infarction comprising the step of:
administering to a subject in need of such treatment an inducible Akt molecule in an amount effective to reduce cardiac tissue necrosis in the subject.

30. A method of modulating tissue damage during transplantation comprising the steps of:
administering to a tissue suspected of tissue damage an expression vector encoding an inducible chimeric protein comprising a mutant Akt polypeptide fused to a ligand-binding domain;
administering to the tissue a second expression vector encoding a second ligand-binding domain fused to a membrane-targeting region; and
modulating tissue damage by administering to the tissue a chemical ligand, wherein the chemical ligand results in activation of the mutant Akt.

31. A method of modulating tissue damage following ischemia-reperfusion comprising the steps of administering to a tissue suspected of tissue damage the fusion protein of claim 7, administering to the tissue a second fusion protein, wherein the second fusion protein comprises a second ligand-binding domain fused to a membrane-targeting region; and modulating tissue damage by administering to the cell a chemical ligand, wherein the chemical ligand results in activation of the mutant Akt.

32. A method of screening compounds to identify a modulator of Akt comprising the steps of:
providing a cell expressing iAkt;
contacting said cell with a candidate compound;
admixing rapamycin analogs to induce activation of Akt;
measuring the level of activation of Akt; and
comparing said Akt activation in the presence of said candidate compound with the activation of Akt in the absence of said candidate compound; wherein a difference in the activation of Akt in the presence of said candidate compound, as compared with the activation of Akt in the absence of said candidate compound, identifies said candidate compound as a modulator of Akt activation.

33. A method of screening compounds to identify a modulator of Akt comprising the steps of:
providing a cell expressing iAkt;
contacting said cell with a candidate compound;
admixing rapamycin analogs to induce activation of Akt;
measuring the level of phosphorylation of Akt; and
comparing said Akt phosphorylation in the presence of said candidate compound with the Akt phosphorylation in the absence of said candidate compound; wherein a difference in the phosphorylation of Akt in the presence of said candidate compound, as compared with the phosphorylation of Akt in the absence of said candidate compound, identifies said candidate compound as a modulator of Akt phosphorylation.

34. A method of screening compounds to identify a modulator of Akt comprising the steps of:
providing a cell expressing iAkt;
contacting said cell with a candidate compound;
admixing rapamycin analogs to induce activation of Akt;
measuring Akt activity; and
comparing said Akt activity in the presence of said candidate compound with the Akt activity in the absence of said candidate compound; wherein a difference in the activity of Akt in the presence of said candidate compound, as compared with the activity of Akt in the absence of said candidate compound, identifies said candidate compound as a modulator of Akt activity.

35. A method of treating a disease by screening compounds to identify a modulator of Akt comprising the steps of:
providing a cell expressing iAkt;
contacting said cell with a candidate compound;
admixing rapamycin analogs to induce activation of Akt;
measuring Akt activity;
comparing said Akt activity in the presence of said candidate compound with the Akt activity in the absence of said candidate compound; wherein a difference in the activity of Akt in the presence of said candidate compound, as compared with the activity of Akt in the absence of said candidate compound, identifies said candidate compound as a modulator of Akt activity; and
administering to a subject suffering from the disease the modulator of Akt activity.

36. The claim of claim 35, wherein the disease is hyperproliferative disease.

37. The claim of claim 36, wherein the hyperproliferative disease is further defined as cancer.

38. The claim of claim 36, wherein the hyperproliferative disease is selected from the group consisting of rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, and psoriasis.

39. The claim of claim 37, wherein the cancer is selected from the group consisting of melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, and colon cancer.

40. A method of activating endogenous Akt comprising the step of administering the fusion protein of claim 7 to a cell.


SEQUENCE LISTING (12 sequences)

SEQ ID NO: 1 - DNA, 2626 bp, mouse
ccgggaccagcggacggaccgagcagcgtcctgcggccggcaccgcggcggcccagatccggccagcagcgcgcgcccggacgccgctgccttcagccggccccgcccagcgcccgcccgcgggatgcggagcggcgggcgcccgaggccgcggcccggctaggcccagtcgcccgcacgcggcggcccgacgctgcggccaggccggctgggctcagcctaccgagaagagactctgatcatcatccctgggttacccctgtctctgggggccacggataccatgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggccgatggactcaagaggcaggaagaagagacgatggacttccgatcaggctcacccagtgacaactcaggggctgaagagatggaggtgtccctggccaagcccaagcaccgtgtgaccatgaacgagtttgagtacctgaaactactgggcaagggcacctttgggaaagtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgcgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggatggtgccactatgaagacattctgcggaacgccggagtacctggcccctgaggtgctggaggacaacgactacggccgtgcagtggactggtgggggctgggcgtggtcatgtatgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctctgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcctgaggcctggggcagcggctggcagctccacgctcctctgcattgccgagtccagaagccccgcatggatcatctgaacctgatgttttgtttctcggatgcgctggggaggaaccttgccagcctccaggaccaggggaggatgtttctactgtgggcagcagcctacctcccagccaggtcaggaggaaaactatcctggggtttttcttaatttatttcatccagtttgagaccacacatgtggcctcagtgcccagaacaattagattcatgtagaaaactattaaggactgacgcgaccatgtgcaatgtgggctcatgggtctgggtgggtcccgtcactgcccccattggcctgtccaccctggccgccacctgtctctagggtccagggccaaagtccagcaagaaggcaccagaagcacctccctgtggtatgctaactggccctctccctctgggcggggagaggtcacagctgcttcagccctagggctggatgggatggccagggctcaagtgaggttgacagaggaacaagaatccagtttgttgctgtgtcccatgctgttcagagacatttaggggattttaatcttggtgacaggagagcccctgccctcccgctcctgcgtggtggctcttagcgggtaccctgggagcgcctgcctcacgtgagccctctcctagcacttgtccttttagatgctttccctctcccgctgtccgtcaccctggcctgtcccctcccgccagacgctggccattgctgcaccatgtcgttttttacaacattcagcttcagcatttttactattataataagaaactgtccctccaaattcaataaaaattgcttttcaagcttgaaaaaaaaaaaaaaaaaaaaaaaaaaaaaaa

SEQ ID NO: 2 - DNA, 1741 bp, mouse
cggctcgcgccgccgccagcactgccgccgttgctgccgccagttcataaataaggagcgggaacgagctcagcgtggcgatgggcgggggtagagcccggccggagaggctgggcggccgccggtgacagacgatactgtatccgaggagcctcctgcatgtcctgctgccctgagctcactcaagctaggtgacagcgtgtgaatgctgccaccatgaatgaggtatctgtcatcaaagaaggctggctccacaaacgtggtgaatacatcaagacctggaggccacggtacttccttctgaagagtgatggatctttcattgggtataaggagaggcccgaggcccctgaccagaccttaccccccctgaacaatttctctgtagcagaatgccagctgatgaagactgagaggccacgacccaacacctttgtcatacgctgcctgcagtggaccacagtcatcgagaggaccttccatgtagactctccagatgagagggaagagtggatgcgggctatccagatggtcgccaacagtctgaagcagcggggcccaggtgaggacgccatggattacaagtgtggctcccccagtgactcttccacatctgagatgatggaggtagctgtcaacaaggcacgggccaaagtgaccatgaatgacttcgattatctcaaactcctcggcaagggcaccttcggcaaggtcattctggttcgagagaaggccactggccgctattatgccatgaagatcctgcgcaaggaggtcatcattgcaaaggatgaagtcgcccacacagtcacagagagccgggttctgcagaataccaggcaccccttccttacagccctcaagtatgccttccagacccatgaccgcctatgctttgtgatggagtatgccaacgggggtgagctgtttttccacctctctcgggagcgagtcttcacggaggatcgggcgcgcttttatggagcagagattgtgtcagctctggagtatttgcactcgagagatgtggtgtaccgtgacatcaagctggaaaaccttatgttggacaaagatggccacatcaagatcactgactttggcttgtgcaaagagggcatcagtgatggagccaccatgaaaaccttctgtggtaccccggagtacttggcgcctgaggtgctagaggacaatgactatgggcgagcagtggactggtgggggctgggtgtggtcatgtatgagatgatgtgtggccgcctgccattctacaaccaggaccacgagcgcctctttgagctcattcttatggaggagatccgcttcccgcgcacactcgggccagaggccaagtccctgctggctggactgctgaagaaggacccaaagcagaggctcggcggaggtcccagtgatgcgaaggaggtcatggagcatagattcttcctcagcatcaactggcaggacgtggtacagaaaaagctcctgccacccttcaaacctcaggtcacttcagaagtggacacaaggtactttgatgacgagttcaccgcccagtccatcacaatcacacccccagaccgatatgacagcctggacccgctggaactggaccagcggacgcacttcccccagttctcctactcagccagcatccgagagtgagcagccctctgccaccacaggacacaagcatggccgtcatccactgcctgggtggctttttaaaaaaaaaaaaaaaaag

SEQ ID NO: 3 - DNA, 1599 bp, human
gagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcgaaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttacccctgcc

SEQ ID NO: 4 - DNA, 2811 bp, human
atgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatatgatgaggatggtatggactgcatggacaatgagaggcggccgcatttccctcaattttcctactctgcaagtggacgagaataagtctctttcattctgctacttcactgtcatcttcaatttattactgaaaatgattcctggacatcaccagtcctagctcttacacatagcaggggcaccttccgacatcccagaccagccaagggtcctcacccctcgccacctttcaccctcatgaaaacacacatacacgcaaatacactccagtttttgtttttgcatgaaattgtatctcagtctaaggtctcatgctgttgctgctactgtcttactattatagcaactttaagaagtaattttccaacctttggaagtcatgagcccaccattgttcatttgtgcaccaattatcatcttttgatcttttagtttttccctcagtgaaggctaaatgagatacactgattctaggtacattttttaactttctagaagagaaaaactaactagactaagaagatttagtttataaattcagaacaagcaattgtggaagggtggtggcgtgcatatgtaaagcacatcagatccgtgcgtgaagtaggcatatatcactaagctgtggctggaattgattaggaagcatttggtagaaggactgaacaactgttgggatatatatatatatatataattttttttttttaaattcctggtggatactgtagaagaagcccatatcacatgtggatgtcgagacttcacgggcaatcatgagcaagtgaacactgttctaccaagaactgaaggcatatgcacagtcaaggtcacttaaagggtcttatgaaacaatttgagccagagagcatctttcccctgtgcttggaaaccttttttccttcttgacatttatcacctctgatggctgaagaatgtagacaggtataatgatactgcttttcaccaaaatttctacaccaaggtaaacaggtgtttgccttatttaattttttactttcagttctacgtgaattagctttttctcagatgttgaaactttgaatgtccttttatgattttgtttatattgcagtagtatttattttttagtgatgagaattgtatgtcatgttagcaaacgcagctccaacttatataaaatagacttactgcagttacttttgacccatgtgcaaggattgtacacgctgatgagaatcatgcactttttctcctctgttaaaaaaaatgataaggctctgaaatggaatatattggttagaatttggctttgggagaagagatgctgccatttaaccccttggtactgaaaatgagaaaatccccaactatgcatgccaaggggttaatgaaacaaatagctgttgacgtttgctcatttaagaatttgaaacgttatgatgacctggcaacaaaaaaaaaaaaaaaaaa

SEQ ID NO: 5 - DNA, 1140 bp, mouse
accgccattcagactgtggccgatggactcaagaggcaggaagaagagacgatggacttccgatcaggctcacccagtgacaactcaggggctgaagagatggaggtgtccctggccaagcccaagcaccgtgtgaccatgaacgagtttgagtacctgaaactactgggcaagggcacctttgggaaagtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgcgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggatggtgccactatgaagacattctgcggaacgccggagtacctggcccctgaggtgctggaggacaacgactacggccgtgcagtggactggtgggggctgggcgtggtcatgtatgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctctgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcc

SEQ ID NO: 6 - DNA, 32 bp, Artificial Sequence
  Primer
agagcgacaacgacgtagccattgtgaaggag

SEQ ID NO: 7 - DNA, 30 bp, Artificial Sequence
  Primer
agagtcgacaccgccattcagactgtggcc

SEQ ID NO: 8 - DNA, 31 bp, Artificial Sequence
  Primer
agagtcgacggctgtgccactggctgagtag

SEQ ID NO: 9 - DNA, 32 bp, Artificial Sequence
  Primer
cgatctcgaggagatgtggcatgaaggcctgg

SEQ ID NO: 10 - DNA, 34 bp, Artificial Sequence
  Primer
cgatgtcgacctttgagattcgtcggaacacatg

SEQ ID NO: 11 - DNA, 48 bp, Artificial Sequence
  Primer
atacaattgccgcggttcgaattctgttttatactcccttcccgtaac

SEQ ID NO: 12 - DNA, 44 bp, Artificial Sequence
  Primer
tatcaattggtttaaacagcaaacagatagataatgagtctcac